(19) United States
(12) Patent Application Publication
(10) Pub. No.: US 2021/0145890 A1
(43) Pub. Date: May 20, 2021

(54) DYSTROPHIN GLYCOPROTEIN COMPLEX SEQUESTERS YAP TO INHIBIT CARDIOMYOCYTE PROLIFERATION

(71) Applicant: Baylor College of Medicine, Houston, TX (US)

(72) Inventors: Yuka Morikawa, Houston, TX (US); Todd Ryan Heallen, Houston, TX (US); John Leach, Houston, TX (US); James F. Martin, Pearland, TX (US)

(21) Appl. No.: 16/616,880

(22) PCT Filed: Jun. 1, 2018
(86) PCT No.: PCT/US18/35697

§ 371 Date: Nov. 25, 2019

Related U.S. Application Data
(60) Provisional application No. 62/513,797, filed on Jun. 1, 2017.

(51) Int. Cl. A61K 35/34; A61K 38/17

(52) U.S. Cl. CPC A61K 35/34; A61K 38/1709

(57) ABSTRACT

Embodiments of the disclosure include methods and compositions related to the proliferation of cardiomyocytes. In particular embodiments, the proliferation of cardiomyocytes is facilitated upon exposure of the cardiomyocytes to agents that affect the sequestering of Yap by dystrophin glycoprotein complex, such agents including those that inhibit DAG1, Yap, or the interaction thereof.

### CROSS-REFERENCE TO RELATED APPLICATIONS

This application claims priority to U.S. Provisional Patent Application Ser. No. 62/513,797, filed Jun. 1, 2017, which is incorporated by reference herein in its entirety.

### STATEMENT REGARDING FEDERALLY SPONSORED RESEARCH OR DEVELOPMENT

This invention was made with government support under DE 023177, HL 127717, HL 130804, and HL 118761 awarded by National Institutes of Health. The government has certain rights in the invention.

### TECHNICAL FIELD

Embodiments of the disclosure concern at least the fields of cell biology, molecular biology, physiology, and medicine.

### BACKGROUND

The regenerative capacity of the adult mammalian heart is limited because of the reduced ability of cardiomyocytes (CMs) to progress through mitosis¹. The regenerative capacity of endogenous CMs exists at birth but is lost postnatally, with subsequent organ growth occurring through CM hypertrophy^{2,3}. The Hippo pathway, a conserved kinase cascade, inhibits CM proliferation in the developing heart to control heart size and in the adult heart to prevent regeneration^{4,5}. The dystrophin glycoprotein complex (DGC), a multicomponent transmembrane complex linking the actin cytoskeleton to extracellular matrix, is essential for CM homeostasis. DGC deficiency in humans results in muscular dystrophy, including lethal Duchenne muscular dystrophy (DMD).

The present application provides solutions to long-felt needs in the art of cardiomuscular repair.

### BRIEF SUMMARY

In particular embodiments, methods and compositions are directed to reversing or overcoming the sequestering of Yap by dystrophin glycoprotein complex that inhibits cardiomyocyte proliferation. In particular aspects, the disclosure concerns inhibition of DAG1, Yap, or binding thereof, using an effective amount of one or more appropriate agents.

Embodiments of the disclosure include a method of regenerating cardiomyocytes, comprising the step of exposing cardiomyocytes to an effective amount of one or more agents that inhibit one or more of the following: (a) DAG1; (b) Yap; (c) the binding of DAG1 to Yap; or (d) the phosphorylation of Yap. In some cases, the agent is a peptide, protein, nucleic acid, small molecule, or combination thereof. The exposing step may occur ex vivo and/or in vivo in a first individual. In at least some cases, when the exposing step occurs ex vivo, the cardiomyocytes are from a first individual and the cardiomyocytes to which the agent(s) are exposed come from the first individual. In certain cases, when the providing step occurs ex vivo, the cardiomyocytes are from the first individual and the cardiomyocytes to which the agent(s) is provided come from a second individual different than the first individual. The first individual may have a muscular (muscular dystrophy, fibrosis, myotonic dystrophy, myocarditis, heart failure, dilated cardiomyopathy, or a combination thereof, for example) or cardiac condition (cardiovascular disease, cardiomyopathy (diabetic cardiomyopathy or age-related cardiomyopathy, as examples), heart failure, myocardial infarction, ischemia, fibrosis, and/or necrosis, for example). The individual may be provided an effective amount of one or more therapies for a muscular condition and/or a cardiac condition.

In one embodiment, there is a method of treating an individual for a muscular or cardiac condition, comprising the step of: (a) providing to the individual an effective amount of cardiomyocytes that have been exposed to one or more agents that inhibit one or more of the following: (1) DAG1; (2) Yap; (3) the binding of DAG1 to Yap; or (4) the phosphorylation of Yap; and/or (b) exposing cardiomyocytes in vivo to one or more of the agents that inhibit one or more of (1), (2), (3), or (4). In some cases, the agent is a peptide, protein, nucleic acid, small molecule, or combination thereof. The exposing step may occur ex vivo and/or in vivo in a first individual. In at least some cases, when the exposing step occurs ex vivo, the cardiomyocytes are from a first individual and the cardiomyocytes to which the agent(s) are exposed come from the first individual. In certain cases, when the providing step occurs ex vivo, the cardiomyocytes are from the first individual and the cardiomyocytes to which the agent(s) is provided come from a second individual different than the first individual. The first individual may have a muscular (muscular dystrophy, fibrosis, myotonic dystrophy, myocarditis, heart failure, dilated cardiomyopathy, or a combination thereof, for example) or cardiac condition (cardiovascular disease, cardiomyopathy (diabetic cardiomyopathy or age-related cardiomyopathy, as examples), heart failure, myocardial infarction, ischemia, fibrosis, and/or necrosis, for example). The individual may be provided an effective amount of one or more therapies for a muscular condition and/or a cardiac condition.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawing, in which:

FIGS. 1A-1P. Combined loss of the dystrophin glycoprotein complex and Hippo pathway in the injured heart. 1A-1D, Trichrome-stained sections of heart apex 21 days after resection. Fibrotic scar (arrowheads) and extra apex (arrows) are denoted. 1E, Fibrotic scar quantification: control (n=9), Mdx (n=4), Salv CKO (n=6), and Salv;Mdx DKO (n=7). Comparisons by analysis of variance (ANOVA) with Bonferroni's multiple comparison test for pairwise comparisons. **p<0.01, ***p<0.001. 1F-1I, Sections showing extra apex (arrows). Dotted lines show resection plane. LV, left ventricle, RV, right ventricle. 1J, Quantification of extra apex size: control (n=9), Mdx (n=4), Salv CKO (n=6), and Salv;Mdx DKO (n=7). Comparisons by ANOVA with Bonferroni's multiple comparison test for pairwise comparisons. *p<0.05, **p<0.01. 1K, Quantification of EdU-positive CMs (n=3 for each). EdU was injected 4 hours before collection. Comparisons by ANOVA with Bonferroni's post-hoc test for pairwise comparisons. *p<0.05, **p<0.01, ***p<0.001. 1L, Quantification of AurkB-positive CMs (n=3 for each). Comparisons by ANOVA with the Mann-Whitney test for pairwise comparisons. *p<0.05, **p<0.01, ***p<0.001. 1M-1P, Immunohistochemical staining of AurkB-positive CMs (arrowheads) Dotted lines delineate dividing CMs. CMs were stained for cardiac troponin T (cTNT), and nuclei with DAPI.

FIGS. 2A-2A′. Yap subcellular localization and expression of downstream targets in Hippo-deficient Mdx cardiomyocytes after apex resection. 2A-2I, Immunohistochemical staining showing Yap localization 4 days after apex resection. CMs were stained cTNT and nuclei with DAPI. Nuclear Yap (arrowheads). 2I, Nuclear Yap quantification in CMs (n=3 for each). Comparisons by two-way ANOVA with Mann-Whitney test for pairwise comparisons. *p<0.05, **p<0.01, ***p<0.001. 2J-2R, Immunohistochemical staining of CCNE2 (arrowheads). r, Quantification of CCNE2-positive CMs (n=3 for each). Comparisons by one-way ANOVA with Tukey post-hoc test for pairwise comparisons. **p<0.01. 2S-2A′, Talin immunohistochemical staining. CMs stained for sarcomeric actinin, and nuclei with DAPI. 2A′, Talin quantification (n=3 for each), measured by pixel intensity. Comparisons by one-way ANOVA with Tukey post-hoc test for pairwise comparisons. *p<0.05, **p<0.01.

FIGS. 3A-3V. Suppression of Mdx cardiomyopathy by Hippo deletion in a pressure overload model. 3A-3N, Transverse aortic constriction (TAC) or sham surgery in 9-week-old control (n=4 for sham, n=5 for TAC), Mdx (n=6 for sham, n=5 for TAC), Salv conditional knockout (CKO; n=4 for sham, n=8 for TAC), and Salv;Mdx double knockout (DKO; n=12 for sham, n=11 for TAC). 3A-3H, Trichrome stained sections 2 weeks after sham (3A-3D) or TAC (3E-3H). 3I, Fibrotic area quantitation. Comparisons by analysis of variance (ANOVA) with Bonferroni's post-hoc test for pairwise comparisons (n=7 for DKO TAC group, n=3 for all others). **p<0.01, ***p<0.001. 3J, 3K, Ejection fraction (EF) and fractional shortening (FS). Detailed analyses are in FIG. 8. Comparisons by ANOVA with Tukey post-hoc test for pairwise comparisons. *p<0.05, **p<0.01. 3L, Estimation of total CM number. Comparisons by ANOVA with Bonferroni's post-hoc test for pairwise comparisons (n=4 for control TAC group, n=8 for DKO TAC group, n=3 for others). *p<0.05, **p<0.01, ***p<0.001. 3M, EdU incorporation in diploid CM nuclei by flow cytometry after TAC. Representative images of flow cytometry are in FIGS. 9A-9L. Comparisons by ANOVA with Tukey post-hoc test for pairwise comparisons (SalvCKO 1 week n=4; n=3 for all others). *p<0.05, **p<0.01. 3N, CM EdU incorporation using tissue sections. Representative images are in FIGS. 9O-9Z. Comparisons by ANOVA with Tukey post-hoc test for pairwise comparisons (n=3 for each). **p<0.01. 3O, Quantification of AurkB positive CMs. Representative images are in FIGS. 10A-10D. Comparisons by ANOVA with Bonferroni's post-hoc test for pairwise comparisons (n=3 for each). *p<0.05, ***p<0.001. 3P, Quantification of nuclear localized Yap in CMs. Representative images are in FIGS. 10E-10L. Groups compared by ANOVA with Bonferroni's post-hoc test for pairwise comparisons (n=3 for each). *p<0.05, **p<0.01, ***p<0.001. 3Q, Active caspase 3-positive CMs quantification at 1 week and 2 weeks after TAC. Representative images are in FIGS. 10M-10T. Comparisons by one-way analysis of variance with Tukey post-hoc test for pairwise comparisons (n=3 for each). *p<0.05, **p<0.01. 3R, 3S, EF and FS in TAC (n=10 for AAV-GFP, n=9 for AAV-Salv) and sham (n=8 for AAV-GFP, n=10 for AAV-Salv). Week 11 comparisons by ANOVA with Tukey post-hoc test for pairwise comparisons. *p<0.05, **p<0.01, ***p<0.001. 3T, Nuclear Yap quantification in CMs. Comparisons by Mann-Whitney U test (n=3 for each). *p<0.05. Representative images in FIGS. 12F-12I. 3U, QRT-PCR of Yap target genes. Comparisons by Mann-Whitney U test (n=5 for each treatment). *p<0.05, **p<0.01. ns, nonsignificant. 3V, Fibrotic area quantification. Comparisons by Mann-Whitney U test (n=5 for each treatment). Representative images in FIGS. 12J, 12K.

FIGS. 4A-4Q. Yap binding to the dystrophin glycoprotein complex. 4A, 4B, Immunoprecipitation (IP) of P12 control and Mdx mouse hearts for Yap (4A) or DAG1 (4B), with immunoblotting of indicated proteins (n=3 for each). 4C, 4D, IP for FLAG(4C) and DAG1(4D) from 6-week-old Yap5SA-expressing hearts with immunoblotting of indicated proteins (n=3 each). 4E-4H, YAP subcellular localization C2C12 cells. Comparisons by ANOVA with Tukey post-hoc test for pairwise comparisons (n=3 for each). **p<0.01. 4I-4P, Deconvolution epifluorescence (super-resolution) microscopic images of Yap subcellular localization in 11-week-old hearts; Cx43, Connexin 43. Yap localization in membrane (arrows) and intercalated discs (arrowhead). 4Q, GST fusion protein binding assay for indicated proteins. DAG1ΔPPxY, DAG1 PPxY motif deletion. Gel source data (FIG. 5).

FIGS. 5A-5O. Cardiomyocyte alignment after apex resection in P8 mouse hearts. 5A-5O, Hearts were collected 21 days after apex resection was performed in P8 mice. Hearts of control (5A, 5E, 5I), Mdx (5B, 5F, 5J), Salv conditional knockout (CKO) (5C, 5G, 5K), and Salv;Mdx double knockout (DKO, 5D, 5H, 5L) mice were stained for cardiac troponin T (cTNT) and wheat germ agglutinin (WGA) for CMs and cell membranes, respectively. For control and Mdx hearts, the apex regions above the scar are shown. For Salv CKO and Salv;Mdx DKO hearts, the apex regions that regenerated are shown. 5M, Sarcomere length in each CM was measured. Groups were compared by using one-way analysis of variance (n=3 for each genotype). No statistically significant difference was observed between samples. 5N, 5O, CM orientation in either the border zone or repaired area. In panel 5N, a representative image shows how the orientation angles of CMs were measured. Angles referenced to the plane of resection were measured for each CM. A total of 50 CMs were measured for each sample. 5O, Histograms showing the distribution of CM orientation angles for each genotype (n=3 for each genotype; 150 CMs total were analyzed for each genotype). Variance between genotype groups was compared by using an F-test.

FIGS. 6A-6R. Protrusion formation in border zone cardiomyocytes and migration of postnatal cardiomyocytes after apex resection in mice. a-e, Apex resection was performed in P8 hearts of control (6A), Mdx (6B), Salv conditional knockout (CKO) (6C), and Salv;Mdx double knockout (DKO, 6D) mice, and hearts were collected 4 days after resection. CMs were stained for cardiac troponin T (cTNT), and images were documented of the tissue around border zone CMs. Dotted lines show plane of resection. Arrowheads show CM protrusions. 6E, Quantification of CM protrusions (n=3 for each genotype). CMs adjacent to the scar were analyzed for length and number of protrusions 4 days after resection. One hundred CMs from each heart were analyzed. Groups were compared by using analysis of variance (ANOVA) with Bonferroni's multiple comparison test for pairwise comparisons. **p<0.01. CM, cardiomyocyte. 6F-6M, Immunostaining for vinculin to visualize cytoskeletal rearrangement in border zone CMs of control (6F, 6J), Mdx (6G, 6K), Salv CKO (6H, 6L), and Salv;Mdx DKO (6I, 6M) mouse hearts. CMs were labeled with anti-sarcomeric actinin. Arrowheads indicate where the upregulation of vinculin is visible in Salv CKO border zone CMs. 6N-6R, Collagen gel assay results for P10 hearts collected from control (6N), Mdx (6O), Salv CKO (6P), and Salv;Mdx DKO mice (6Q). Gel was stained with DAPI and for cTNT. 6R, Quantification of hearts in which migration was observed. Groups were compared by using the Fisher's exact test. Control vs Salv CKO, p=0.0097; Mdx vs Salv CKO, p=0.0097; Salv CKO vs Salv;Mdx DKO, p=0.007.

FIGS. 7A-7G Characterization of mouse hearts after transverse aortic constriction surgery. 7A-7E, Knockout efficiency in Salv conditional knockout (CKO) mice. Immunohistochemical analysis of Salv was performed in control (7A, 7C) and Salv CKO (7B, 7D) mouse hearts 2 weeks after transverse aortic constriction (TAC) surgery. CMs were labeled with anti-sarcomeric actinin. 7E, Quantification of Salv intensity (n=3 for each treatment) measured according to pixel intensity. Groups were compared by using the Mann-Whitney U test (n=3 for each treatment). *p<0.05. 7F-7G Histology and cell size after TAC surgery. 7F, Representative images showing trichrome staining of hearts 2 weeks after TAC surgery in control Mdx, Salv CKO, and Salv;Mdx double knockout (DKO) mice. 7G, CM size 2 weeks after sham or TAC surgery. Cell size was measured in wheat germ agglutinin (WGA)-stained sections by using ImageJ software. Groups were compared by using analysis of variance (ANOVA) with Bonferroni's post-hoc test for pairwise comparisons (n=3 each). *p<0.05, **p<0.01, ***p<0.001.

FIGS. 8A-8E Echocardiographic measurements. 8A, Color Doppler echocardiography across the transverse aorta before transverse aortic constriction (pre-TAC, left panel), and after transverse aortic constriction (post-TAC, right panel). The constriction site (TAC) is labeled on the post-TAC image. 8B, Doppler echocardiography measurement of peak pressure through the constriction site two weeks after TAC. Sham (n=9), TAC (n=7). Groups were compared by using analysis of variance (ANOVA) with the Tukey post-hoc test for pairwise comparisons, ***p<0.001. 8C, Interventricular septal (IVS) thickness during diastole (IVS.d, left panel) and systole (IVS.s, right panel). 8D, Left ventricular internal diameter (LVID) during diastole (LVID.d, left panel) and systole (LVID.s, right panel). 8E, Left ventricular posterior wall (LVPW) thickness during diastole (LVPW.d, left panel) and systole (LVPW.s, right panel). Sham control (n=4); sham Salv conditional knockout (CKO, n=4); sham Mdx (n=6); sham Salv;Mdx double knockout (DKO, n=12). TAC control (n=5); TAC Salv CKO (n=8); TAC Mdx (n=5); TAC Salv;Mdx DKO (n=11). Groups were compared by using analysis of variance (ANOVA) with the Tukey post-hoc test for pairwise comparisons. Significant differences with the Mdx group two weeks post TAC are indicated. *p<0.05, **p<0.01, ***p<0.001.

FIGS. 9A-9Z. EdU incorporation analysis after transverse aortic constriction (TAC) surgery. 9A-9N. Flow cytometry analysis on isolated nuclei after transverse aortic constriction (TAC) surgery. 9A-9L, Representative images of flow cytometry analysis on the nuclei isolated from control (9A, 9E, 9I), Mdx (9B, 9F, 9J), Salv conditional knockout (CKO; 9C, 9G, 9K), and Salv;Mdx double knockout (DKO; 9D, 9H, 9L) mouse hearts after TAC surgery. 9A-9D, PCM1+ population was gated and plots show EdU incorporation. 9E-9H, Histogram showing DAPI intensity in PCM1+ population and discrimination between 2N, 4N, and >4N population. 9I-9L, Histogram showing DAPI intensity in PCM1+, EdU+ population. 9M, 9N, Quantification of PCM1+, EdU+ nuclei in >2N-4N (m) and >4N (n) population. Groups were compared by using analysis of variance (ANOVA) with the Tukey post-hoc test for pairwise comparisons (n=3). *p<0.05, **p<0.01. 9O-9V, Representative images showing EdU staining of control (9O, 9U), Mdx (9P, 9V), peri-fibrotic area of Mdx (9Q, 9W), Salv conditional knockout (CKO) (9R, 9X), Salv;Mdx double knockout (DKO; 9S,9Y) and peri-fibrotic area of DKO (9T, 9Z) mouse heart sections after TAC surgery collected after 2 weeks. CMs were stained for actinin and cells were delineated by WGA. Arrowheads show EdU-positive CMs. Quantification of EdU-positive CMs is shown in FIG. 3N. Peri-fibrotic area defined in Methods.

FIGS. 10A-10T. Immunohistochemical analysis of mouse hearts after transverse aortic constriction (TAC) surgery. 10A-10D, Representative images for aurora kinase B (AurkB) staining of control (10A), Mdx (10B), Salv CKO (10C), and Salv;Mdx DKO (10D) mouse hearts 2 weeks after TAC surgery. CMs were stained with anti-cTNT antibody. Quantification of AurkB-positive CMs is shown in FIG. 3O. Arrowheads indicate positive AurkB staining. 10E-10K, Representative images showing Yap staining of control (10E, 10I), Mdx (10F, 10J), Salv conditional knockout (CKO) (10G, 10K), and Salv;Mdx double knockout (DKO; 10H, 10L) mouse hearts after TAC surgery. CMs were detected by immunostaining for cardiac troponin T (cTNT). Arrowheads point to Yap localized in nuclei. Quantification of CMs with nuclear Yap is shown in FIG. 3P. 10M-10T, Representative images for active caspase 3 staining of control (10M, 10Q), Mdx (10N, 10R), Salv conditional knockout (CKO; 10O, 10S), and Salv;Mdx double knockout (DKO; 10P, 10T) mouse hearts 1 and 2 weeks after TAC surgery. CMs were stained with anti-cardiac troponin T (cTNT) antibody. Arrowheads show active caspase 3-positive CMs. Quantification of active caspase 3-positive CMs is shown in FIG. 3Q.

FIGS. 11A-11Q. Immunohistochemical analysis for phospho-Yap and Vinculin after transverse aortic constriction (TAC) surgery. 11A-11H, Representative images showing phospho-Yap (P-Yap) staining of control (11A, 11E), Mdx (11B, 11F), Salv CKO (11C, 11G), and Salv;Mdx DKO (11D, 11H) mouse hearts after TAC surgery. CMs were detected by immunostaining for cardiac troponin T (cTNT). Arrows indicate P-Yap in intercalated discs. 11I, Quantification of P-Yap levels (n=3 for each genotype) measured according to pixel intensity. Groups were compared by using one-way analysis of variance with the Tukey post-hoc test for pairwise comparisons. **p<0.01. 11J-11Q, Representative images for vinculin staining of control (11J, 11N), Mdx (11K, 11O), Salv CKO (11L, 11P), and Salv;Mdx DKO (11M, 11Q) mouse hearts after TAC surgery. Vinculin staining was used to detect cytoskeletal rearrangements in CMs after TAC surgery. Sarcomeres were stained with anti-sarcomeric actinin.

FIGS. 12A-12K. Adeno-associated virus (AAV9)-treated Mdx hearts after transverse aortic constriction (TAC) surgery. Mouse hearts were collected 11 weeks after TAC surgery. 12A-12D, Staining for Salvador in Mdx hearts transfected with AAV9-GFP (12A, 12B) or AAV9-Salv (12C, 12D). CMs were stained with actinin. 12E, Quantification of Salv (n=5 each treatment) measured according to pixel intensity. Groups were compared by using the Mann-Whitney U test. **p<0.01. 12F-12I, Staining for Yap in Mdx mouse hearts transfected with AAV-GFP (12F, 12G) or AAV-Salv (12H, 12I). Arrowheads point to Yap localized in nuclei. 12J, 12K, Representative images showing trichrome staining of Mdx mouse hearts transfected with AAV9-GFP (12J) or AAV9-Salv (12K).

FIGS. 13A-13D. Immunoprecipitation and subcellular localization studies in C2C12 cells. 13A, Knockdown efficiency of the small interfering (si)RNAs used in this study. C2C12 cells were differentiated and treated with siSalv, siDmd, or control siRNA for 48 hours before harvesting. Groups were compared by using the Mann-Whitney U test (n=3 for each treatment). *p<0.05. 13B-13D, Immunoprecipitation was performed by using protein extracts of differentiated C2C12 cells with antibodies specific for Yap (13B), FLAG (13C), or DAG1 (13D), followed by immunoblotting of indicated proteins. Yap5SA or green fluorescent protein (GFP) constructs were transfected into C2C12 cells before differentiation (13C, 13D). For gel source data, see FIG. 5.

FIG. 14. Model of interaction between the Hippo pathway and the dystrophin glycoprotein complex (DGC). ICD, intercalated disc.

(Left Side)
1. Hippo signaling is low. YAP phosphorylation and YAP binding to the DGC is reduced.
2. YAP-TEAD promote the transcription of target genes including Sgcδ and α-catenin.
3. YAP-TEAD promote DGC assembly by promoting the expression of the core component Sgcδ.
4. The ICD is immature in neonatal cardiomyocytes. Yap promotes the expression of the ICD component α3-catenin.

(Right Side)
1. Hippo signaling is high. YAP phosphorylation and YAP binding to the DGC is increased.
2. YAP-TEAD transcription-activating activity is reduced.
3. The DGC sequesters phosphorylated YAP through an interaction involving the PPxY motif of DAG1.
4. The ICD is mature in adult cardiomyocytes. YAP is incorporated into the ICD independent of Hippo through α-catenin binding.

### DETAILED DESCRIPTION

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “therapeutically effective amount” is synonymous with “therapeutically effective dose” or “effective dose” and refers to the amount of compound that will elicit the biological or clinical response being sought by the practitioner in an individual in need thereof. As a non-limiting example, an effective amount is an amount sufficient to regenerate cells or tissue, including that related to cardiomyocytes.

The DGC component dystroglycan 1 (DAG1) directly binds to Hippo pathway effector Yap to inhibit CM proliferation. As shown herein, the Yap-DAG1 interaction was enhanced by Hippo-induced Yap phosphorylation, revealing a connection between Hippo pathway function and the DGC. After injury, Hippo-deficient postnatal hearts maintained organ size control by repairing the defect with correct dimensions, whereas postnatal hearts doubly deficient for Hippo and the DGC showed CM overproliferation at the injury site. In mature Mdx mouse hearts—a model of DMD—Hippo deficiency protected against overload-induced heart failure.

### I. Compositions

Embodiments of the disclosure include compositions comprising one or more agents that inhibit one or more of DAG1, Yap, the binding of DAG1 to Yap, and/or the phosphorylation of Yap. Also included are cells that were exposed to one or more agents that inhibit one or more of DAG1, Yap, the binding of DAG1 to Yap, and/or the phosphorylation of Yap. The cells may be cardiomyocytes or cells that are induced to differentiate into cardiomyocytes. Mixtures of the agents and/or cells with the agents are encompassed in the disclosure.

Although the agent may be of any kind, in specific cases the agent comprises a peptide, protein, nucleic acid, small molecule, cells, or a combination thereof.

In cases wherein the agent is a peptide, the peptide may be of any kind and/or length so long as it is capable of the intended inhibition. The peptide may be at least or no more than 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, and so forth amino acids in length. The peptide may have a mutated site compared to a corresponding wildtype region.

The peptide may or may not be a fragment of DAG1 or Yap. In cases wherein the peptide is a fragment of Yap, the fragment may or may not include one or more phosphorylation sites of Yap. In cases wherein the peptide includes at least one phosphorylation of Yap, the fragment may include one or more of serine residues that are phosphorylated in wild type, including those phosphorylated by Lats, for example. Examples of phosphorylation sites that may be included in the peptide are serines such as S61, S127, S128, S131, S163, S164, and S381.

In cases wherein the agent is a protein, the protein may be of any kind or length. The protein may or may not comprise one or more mutations with respect to a corresponding wildtype protein. The protein may or may not be an antibody or active fragment thereof. In specific embodiments, the antibody binds Yap or DAG1. As used herein, the term “antibody” is intended to refer broadly to any immunologic binding agent such as IgG, IgM, IgA, IgD and IgE. The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

In some embodiments, an inhibitor is utilized that inhibits a Yap or DAG1 nucleic acid. In specific embodiments, nucleic acid that targets expression of Yap or DAG1 are utilized as the agent(s). In some cases shRNA or siRNA are utilized that inhibit any region of a Yap or DAG1 mRNA. The disclosure includes at least one vector. The vector comprises a) an eukaryotic promoter; b) at least one polynucleotide encoding a small hairpin RNA (shRNA), the polynucleotide comprising a nucleotide sequence that corresponds to a nucleotide sequence in a Yap mRNA transcript and/or a DAG1 mRNA transcript.

As used herein, the terms “small interfering” or “short interfering RNA” or “siRNA” refer to an RNA duplex of nucleotides that is targeted to a desired gene and is capable of inhibiting the expression of a gene with which it shares homology. The RNA duplex comprises two complementary single-stranded RNAs of 15, 16, 17, 18, 19, 20, 21, 22, 23, 24, or 25 nucleotides that form 15, 16, 17, 18, 19, 20, 21, 22, 23, 24, or 25 base pairs and possess 3′ overhangs of two nucleotides. The RNA duplex is formed by the complementary pairing between two regions of a RNA molecule. siRNA is “targeted” to a gene in that the nucleotide sequence of the duplex portion of the siRNA is complementary to a nucleotide sequence of the targeted gene. In some embodiments, the length of the duplex of siRNAs is less than 30 nucleotides. The duplex can be 29, 28, 27, 26, 25, 24, 23, 22, 21, 20, 19, 18, 17, 16, 15, 14, 13, 12, 11 or 10 nucleotides in length. The length of the duplex can be 17-25 nucleotides in length. The duplex RNA can be expressed in a cell from a single construct.

As used herein, the term “shRNA” (small hairpin RNA) refers to an RNA duplex wherein a portion of the siRNA is part of a hairpin structure (shRNA). In addition to the duplex portion, the hairpin structure may contain a loop portion positioned between the two sequences that form the duplex. The loop can vary in length. In some embodiments the loop is 5, 6, 7, 8, 9, 10, 11, 12 or 13 nucleotides in length. The hairpin structure can also contain 3′ or 5′ overhang portions. In some aspects, the overhang is a 3′ or a 5′ overhang 0, 1, 2, 3, 4 or 5 nucleotides in length. In one aspect of this invention, a nucleotide sequence in the vector serves as a template for the expression of a small hairpin RNA, comprising a sense region, a loop region and an antisense region. Following expression the sense and antisense regions form a duplex. It is this duplex, forming the shRNA, which hybridizes to, for example, the Yap mRNA or DAG1 mRNA and reduces expression of Yap or DAG1 respectively.

As used herein, the term “vector” refers to any viral or non-viral vector, as well as any plasmid, cosmid, phage or binary vector in double or single stranded linear or circular form that may or may not be self-transmissible or mobilizable, and that can transform prokaryotic or eukaryotic host cells either by integration into the cellular genome or which can exist extrachromosomally (e.g., autonomous replicating plasmid with an origin of replication). Any vector known in the art is envisioned for use in the practice of embodiments of this disclosure.

In specific embodiments, a siRNA or shRNA targets a Yap mRNA or a DAG1 mRNA. An example of a Yap mRNA sequence to target is in NCBI GenBank® Accession No. AB567721 (human) and BC094313 (mouse), and an example of a DAG1 mRNA sequence to target is in GenBank L19711 (human) and BC007150 (mouse), all of which are incorporated by reference herein in their entirety

### II. Examples of Methods of Use

In particular embodiments there are methods of treating a medical condition by delivering a therapeutically effective amount of (a) one or more agents that inhibit one or more of DAG1, Yap, the binding of DAG1 to Yap, and/or the phosphorylation of Yap to the individual and/or (b) cardiomyocytes that were exposed to one or more agents that inhibit one or more of DAG1, Yap, the binding of DAG1 to Yap, and/or the phosphorylation of Yap. At least one symptom of any medical condition may be improved upon administration of one or more of the agents.

Certain embodiments of the disclosure include methods of regenerating cardiomyocytes in an individual by providing to the individual a therapeutically effective amount of (a) one or more agents that inhibit one or more of DAG1, Yap, the binding of DAG1 to Yap, and/or the phosphorylation of Yap to the individual and/or (b) cardiomyocytes that were exposed to one or more agents that inhibit one or more of DAG1, Yap, the binding of DAG1 to Yap, and/or the phosphorylation of Yap.

Certain methods may be performed on one or more individuals that are in need of regeneration of cardiomyocytes, such as individuals with damage to the heart or at risk for damage to the heart. The individual may have damaged heart muscle tissue in need of regeneration of cardiomyocytes. In particular embodiments, the administration of one or more of the agents results in regeneration of cardiomyocytes from existing cardiomyocytes in the individual.

Embodiments of the disclosure include methods and/or compositions for regeneration of cardiac muscle and reversal of myocardial ischemic injury, for example. In particular embodiments, there are methods for stimulating proliferation of resident adult cardiac progenitor or cardiac muscle cells in mammalian hearts that have had a cardiac condition, such as acute ischemic injury, for example. In certain embodiments, such methods are achieved with compositions comprising (a) one or more agents that inhibit one or more of DAG1, Yap, the binding of DAG1 to Yap, and/or the phosphorylation of Yap to the individual and/or (b) cardiomyocytes that were exposed to one or more agents that inhibit one or more of DAG1, Yap, the binding of DAG1 to Yap, and/or the phosphorylation of Yap.

Embodiments of the present disclosure are directed to methods and/or compositions related to therapy and/or prevention of one or more cardiac conditions. Embodiments of the present disclosure concern regeneration of tissue, including muscle tissue, such as myocardial tissue. Certain embodiments relate to reversal of a cardiac condition (or improvement of at least one symptom thereof), including at least heart disease, cardiomyopathy, heart valve problems, pericarditis, arrhythmia, cardiac arrest, congenital heart defect, heart failure, cardiac disease, cardiotoxicity, congestive heart failure, ischemic heart disease, acute myocardial infarction, atrial fibrillation, coronary artery disease, ischemic heart disease, valvular heart disease, hypertensive heart disease, and arrhythmias. Particular types of cardiovascular disease may be treated or prevented, such as coronary artery disease (also known as coronary heart disease and ischaemic heart disease); cardiomyopathy (diseases of cardiac muscle); heart failure; cor pulmonale; cardiac dysrhythmias; inflammatory heart disease; endocarditis; inflammatory cardiomegaly; myocarditis; valvular heart disease; cerebrovascular disease; peripheral arterial disease; congenital heart disease; and rheumatic heart disease. Particular but exemplary indications of embodiments of the disclosure include at least applications for 1) congestive heart failure; 2) prevention from ventricular remodeling or aneurysm of myocardial infarction; and/or 3) cardiomyopathy. In specific embodiments, methods and compositions of the disclosure provide cardiomyocyte regeneration that is sufficient to reverse established cardiac condition or prevention of a cardiac condition or delay of onset or reduction in severity.

Embodiments of the disclosure include delivery of one or more of the agents to stimulate regeneration of cells (such as muscle cells, including cardiomyocytes) and/or tissue (including cardiac tissue). Particular aspects for such embodiments result in reversal of one or more cardiac-related medical conditions. Certain aspects for such embodiments result in improvement of at least one symptom of a medical condition, such as a cardiac-related medical condition.

In some cases, methods and compositions of the present disclosure are employed for prevention of one or more cardiac-related medical conditions or delay of onset of one or more cardiac-related medical conditions or reduction of extent of one or more symptoms of one or more cardiac-related medical conditions. In particular cases, such prevention, delay or onset, or reduction of extent of one or more symptoms, occurs in an individual that is at risk for a cardiac-related medical condition. Exemplary risk factors include one or more of the following: age, gender (male, although it occurs in females), high blood pressure, high serum cholesterol levels, tobacco smoking, excessive alcohol consumption, sugar consumption, family history, obesity, lack of physical activity, psychosocial factors, diabetes mellitus, overweight, genetic predisposition, and/or exposure to air pollution.

Any individual being treated may be an adult, adolescent, child, infant, or the treatment may be in utero.

Delivery to the individual of the one or more agents may be systemic or may be local. In specific examples, following use of the agent(s), in vitro-derived cardiomyocytes are delivered to an individual. In some cases, the agent(s) is delivered in vivo within the heart.

In cases wherein cells are delivered as the therapeutic agent, one may use one or more agents to expand patient-derived (or non-patient-derived) induced-pluripotent stem cell-derived cardiomyocytes (or embryonic stem cell-derived cardiomyocytes) in culture before transplanting into a patient. In such cases, cardiomyocytes are produced from non-cardiomyocyte cells, such as stem cells of any kind, including induced pluripotent stem cells or embryonic stem cells, and an effective amount of the one or more agents may be exposed to the cells during and/or after the production method of producing cardiomyocytes from the stem cells. In some cases, the one or more agents are utilized to expand stem cell-derived cardiomyocytes after transplanting into the patient (for example, using small-molecule driven promoters), or other Yap induction schemes engineered into the stem-cell derived cardiomyocytes. In certain cases, a small-molecule driven promoter will be used to direct expression of nucleic acids that reduce expression of Hippo pathway components such as Yap, Salvador and/or DAG1. This approach may be used to transiently modulate Yap activity in the induced-pluripotent stem-cell derived cardiomyocytes.

In one aspect of the disclosure, the disclosure is directed to compositions and methods for selectively reducing the expression of the gene product from the Yap or DAG1 gene in a eukaryotic cell, as well as for treating diseases in mammals, such as for example, but not limited to, humans, mice and rats, caused by the expression of the gene. The present disclosure provides a vector comprising a polynucleotide sequence which comprises a nucleic acid sequence encoding a small interfering RNA molecule (siRNA) targeted against the Yap or DAG1 gene. The siRNA forms a hairpin structure comprising a duplex structure and a loop structure. The loop structure may contain from 4 to 10 nucleotides, such as 4, 5, 6, 7, 8, 9, 10 nucleotides. The duplex is less than 30 nucleotides in length, such as from 10 to 27 nucleotides. The siRNA may further comprise an overhang region. Such an overhang may be a 3′ overhang region or a 5′ overhang region. The overhang region may be, for example, 1, 2, 3, 4, 5, 6 nucleotides in length.

An individual receiving the therapy encompassed herein may or may not have been diagnosed with a medical condition, including a cardiac medical condition, for example. The individual may or may not be exhibiting one or more symptoms of having a cardiac medical condition without having a previous diagnosis, for example.

### III. Pharmaceutical Preparations

Pharmaceutical compositions of the present invention comprise an effective amount of (a) one or more agents that inhibit one or more of DAG1, Yap, the binding of DAG1 to Yap, and/or the phosphorylation of Yap to the individual and/or (b) cardiomyocytes that were exposed to one or more agents that inhibit one or more of DAG1, Yap, the binding of DAG1 to Yap, and/or the phosphorylation of Yap, said agents and/or cells dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that of the agents will be known to those of skill in the art in light of the present disclosure, as exemplified by Remington: The Science and Practice of Pharmacy, 21^{st} Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The one or more agents may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The one or more agents may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present disclosure, the composition is combined with the carrier in any convenient and practical manner, i.e., by solution, suspension, emulsification, admixture, encapsulation, absorption and the like. Such procedures are routine for those skilled in the art.

In a specific embodiment of the present disclosure, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present disclosure may concern the use of a pharmaceutical lipid vehicle compositions that include the one or more agents, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the one or more agents may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

A. Alimentary Compositions and Formulations

In preferred embodiments of the present invention, the one or more agents are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

B. Parenteral Compositions and Formulations

In further embodiments, the one or more agents, or the cells, may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

C. Miscellaneous Pharmaceutical Compositions and Formulations

In other preferred embodiments of the invention, the active compound may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### IV. Kits of the Disclosure

Any of the compositions described herein may be comprised in a kit. In a non-limiting example, (a) one or more agents that inhibit one or more of DAG1, Yap, the binding of DAG1 to Yap, and/or the phosphorylation of Yap to the individual and/or (b) cardiomyocytes that were exposed to one or more agents that inhibit one or more of DAG1, Yap, the binding of DAG1 to Yap, and/or the phosphorylation of Yap, may be comprised in a kit.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the one or more compositions in close confinement for commercial sale. Such containers may include injection or blow-molded plastic containers into which the desired vials are retained.

The composition may be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit. However, the components of the kit may be provided as dried powder(s). When reagents and/or components are provided as a dry powder, the powder can be reconstituted by the addition of a suitable solvent. It is envisioned that the solvent may also be provided in another container means.

The kits of the present disclosure will also typically include a means for containing the vials in close confinement for commercial sale, such as, e.g., injection and/or blow-molded plastic containers into which the desired vials are retained.

In particular embodiments, the kit comprises reagents and/or tools for determining that an individual has a cardiac-related medical condition. In some embodiments, the kit comprises one or more additional therapies for a cardiac-related medical condition, such as one or more of ACE Inhibitor, aldosterone inhibitor, angiotensin II receptor blocker (ARBs); beta-blocker, calcium channel blocker, cholesterol-lowering drug, digoxin, diuretics, inotropic therapy, potassium, magnesium, vasodilator, anticoagulant medication, aspirin, and a combination thereof.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the subject matter of the disclosure.

### Example 1

### Dystrophin Glycoprotein Complex Sequesters Yap to Inhibit Cardiomyocyte Proliferation

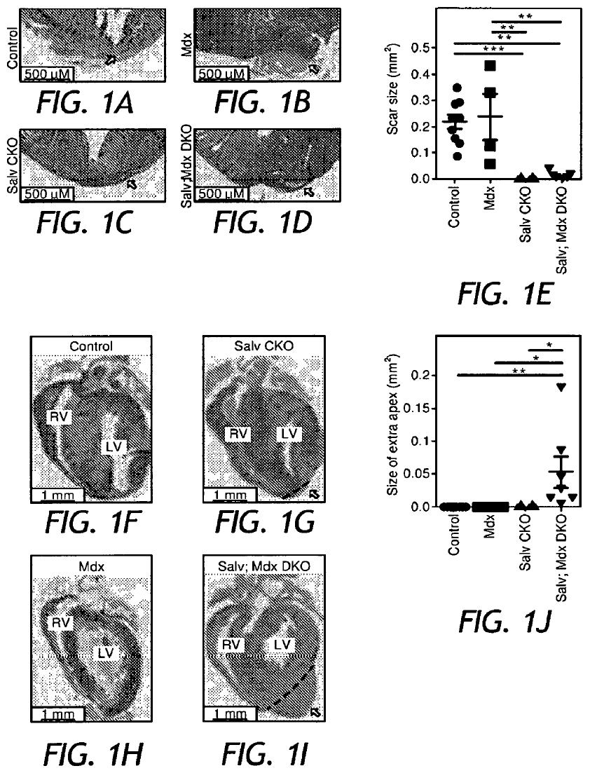

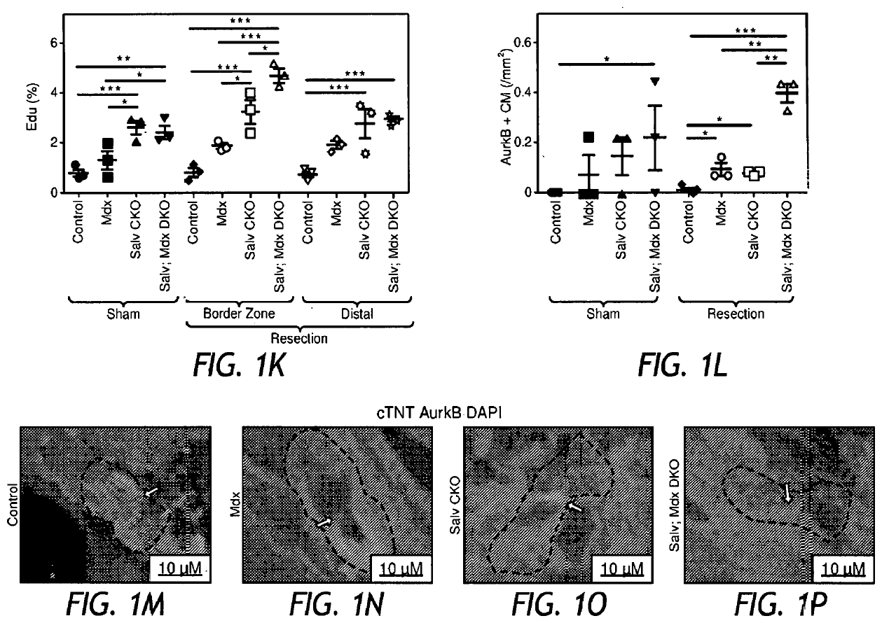

Previous work revealed that DGC components are Yap targets⁶. To explore the connection between the Hippo pathway and DGC function, the inventors conditionally deleted the gene encoding the Hippo pathway component Salvador (Salv) in myocardium in an Mdx (dystrophin loss of function) background^{4,7}. Neonatal cardiac apex resections were performed at nonregenerative postnatal (P) day 8, and resected hearts collected at P29 revealed that control and Mdx hearts failed to regenerate (FIGS. 1A, 1B, 1E, 1F, 1H), whereas Salv conditional knockout (CKO) hearts regenerated efficiently (FIGS. 1C, 1E, 1G). Remarkably, Salv;Mdx double knockout (DKO) hearts regenerated with excessive myocardial growth at the resection site, often with a completely formed secondary cardiac apex (FIGS. 1D, 1E, 1I, 1J). Both Salv CKO and Salv;Mdx DKO resected hearts had reduced scarring, indicating efficient cardiac repair (FIGS. 1C, 1D, 1E).

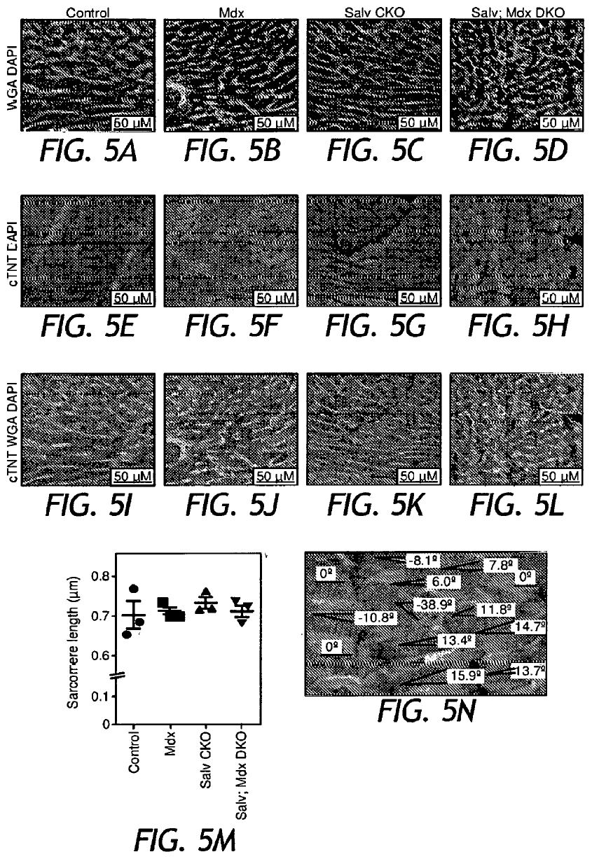

Immunofluorescence studies in Salv;Mdx DKO secondary apex CMs 21 days after resection revealed that CM length was unchanged when compared to that in other groups (FIGS. 5A-5M). In the Salv CKO, regenerating CMs displayed an organized alignment when measured against the resection plane⁸; however, Salv;Mdx DKO CMs had a broad range of orientations and appeared disorganized (FIGS. 5N, 5O), indicating that CM outgrowth in the Salv;Mdx DKO secondary apex was uncoordinated. This overgrowth phenotype suggests that the Hippo pathway and DGC cooperatively limit tissue growth after injury and that DGC function is required for the normal three-dimensional organization of regenerating CMs.

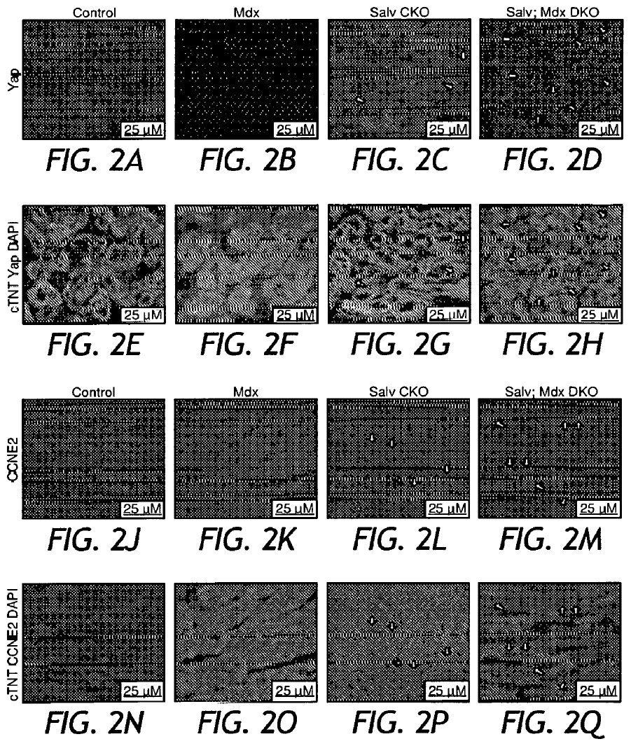

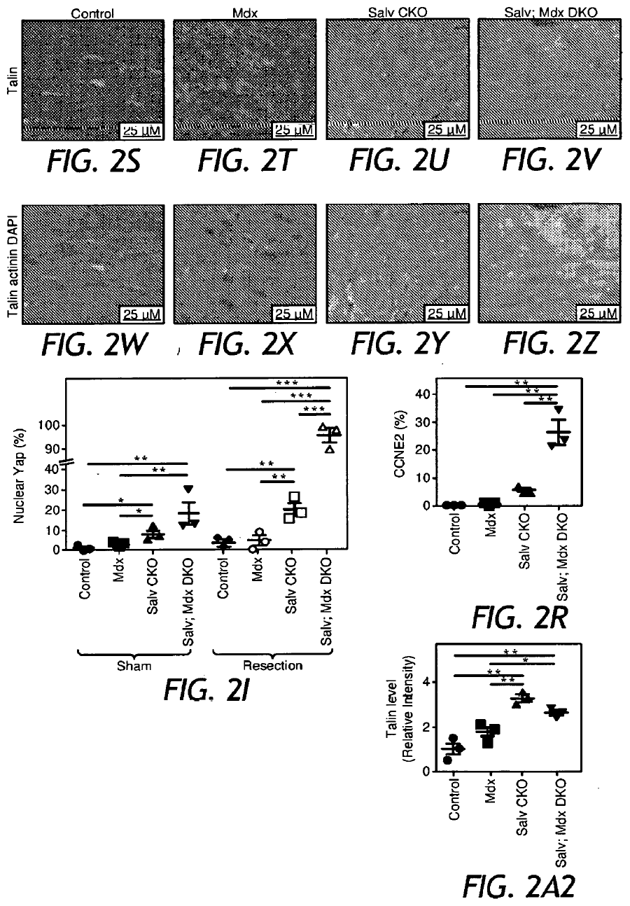

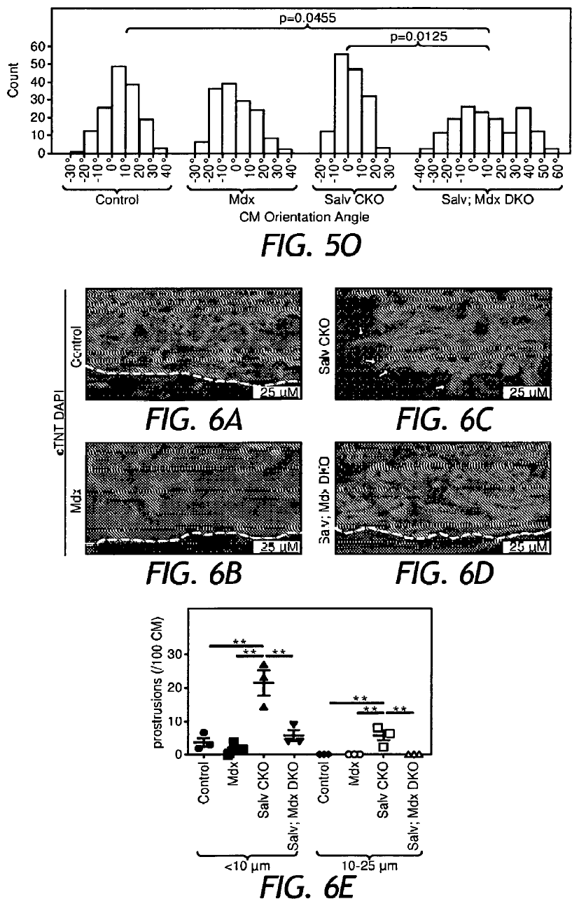

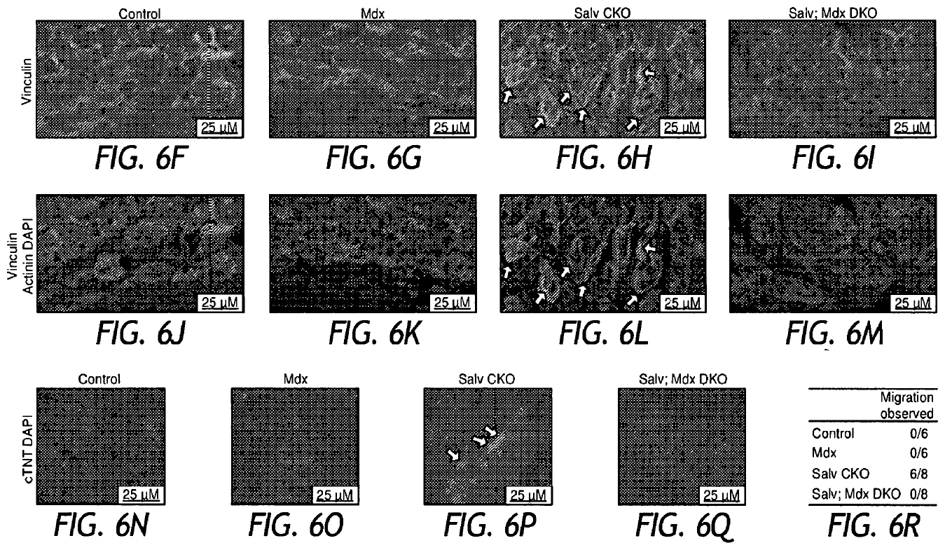

There are cellular protrusions in regenerating Salv CKO border zone CMs⁶. In regenerative stage hearts, when Hippo activity is low, protrusion formation promoted regeneration; however Mdx CMs failed to make protrusions and regenerate, indicating the requirement for dystrophin⁶. It was determined whether protrusion formation could be rescued after injury at the non-regenerative stage in Salv;Mdx DKO adult hearts. Whereas Salv CKO border zone CMs formed protrusions, Salv;Mdx DKO CMs failed to make protrusions (FIGS. 2A-2E). Similarly, vinculin expression was elevated in Salv CKO border zone CMs but not in those of other groups (FIGS. 6F-6M). In vitro migration studies also revealed that dystrophin deficiency in Salv;Mdx DKO hearts suppressed the migratory phenotype of Salv CKO CMs (FIGS. 6N-6R), suggesting that the disorganized morphology of regenerated CMs in Salv;Mdx DKO hearts was due to lack of protrusion formation.

DNA synthesis and expression of the mitosis and cytokinesis marker aurora kinase B (Aurkb) were increased in injured Salv;Mdx DKO CMs compared to injured control CMs (FIGS. 1K, 1L), indicating enhanced myocardial proliferation in Salv;Mdx DKO hearts. After apex resection, Salv CKO and Salv;Mdx DKO CMs showed increased cytokinesis compared to control CMs (FIGS. 1L-1P). Similar to DNA synthesis, cytokinesis was increased in Salv;Mdx DKO CMs compared to Salv CKO CMs. These results indicate that the Hippo pathway and DGC cooperatively inhibit CM cell cycle progression.

To investigate whether the DGC regulates Yap by altering its subcellular localization after injury, Yap localization was examined in border zone CMs after apex resection. The number of CMs with nuclear Yap was low in control and Mdx hearts (FIGS. 2A, 2B, 2E, 2F, 2I) but was elevated in Salv CKO hearts (FIGS. 2C, 2G, 2I), as reported previously⁶. In Salv;Mdx DKO hearts, Yap was localized in the nucleus of most CMs (FIGS. 2D, 2H, 2I). Furthermore, the expression of Yap targets Ccne2 and Talin was upregulated in Salv CKO and Salv;Mdx DKO CMs (FIGS. 2J-2A′). These results suggest that the Hippo pathway and DGC cooperatively inhibit Yap nuclear localization to prevent Yap transcriptional activity.

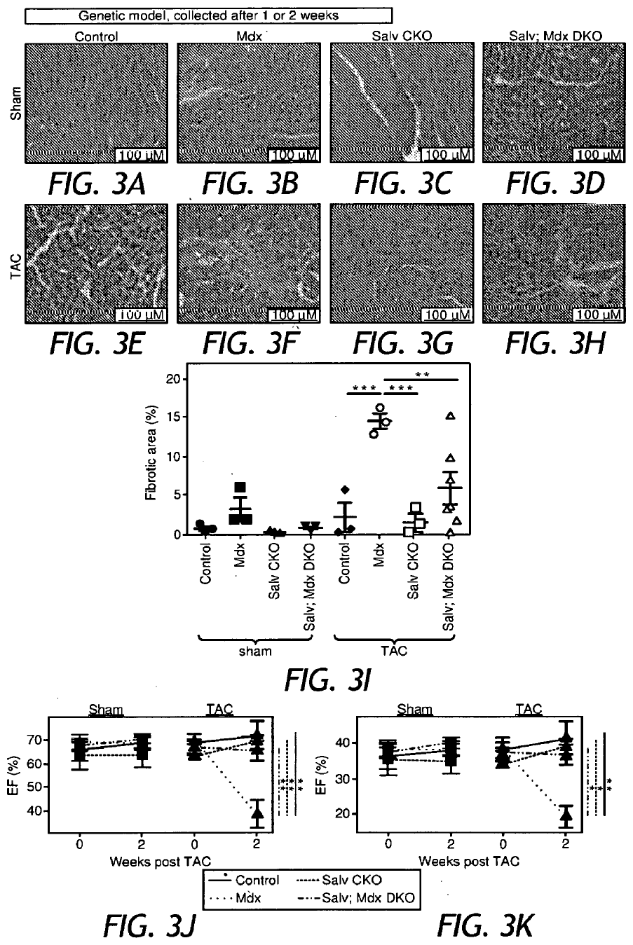

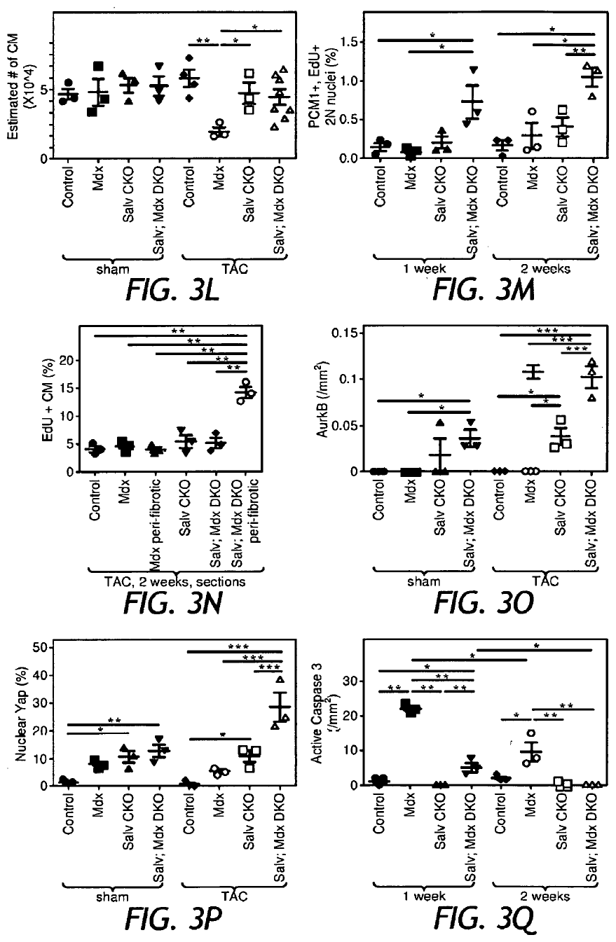

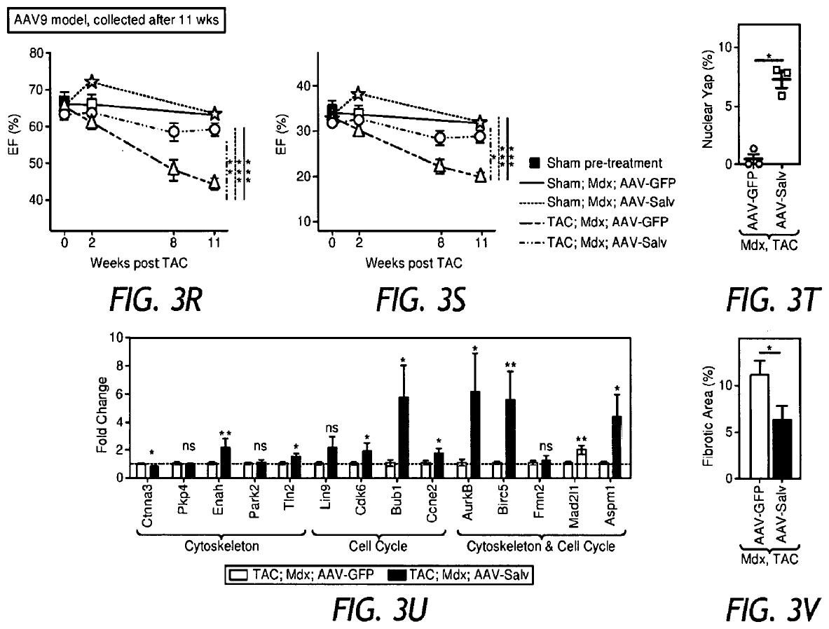

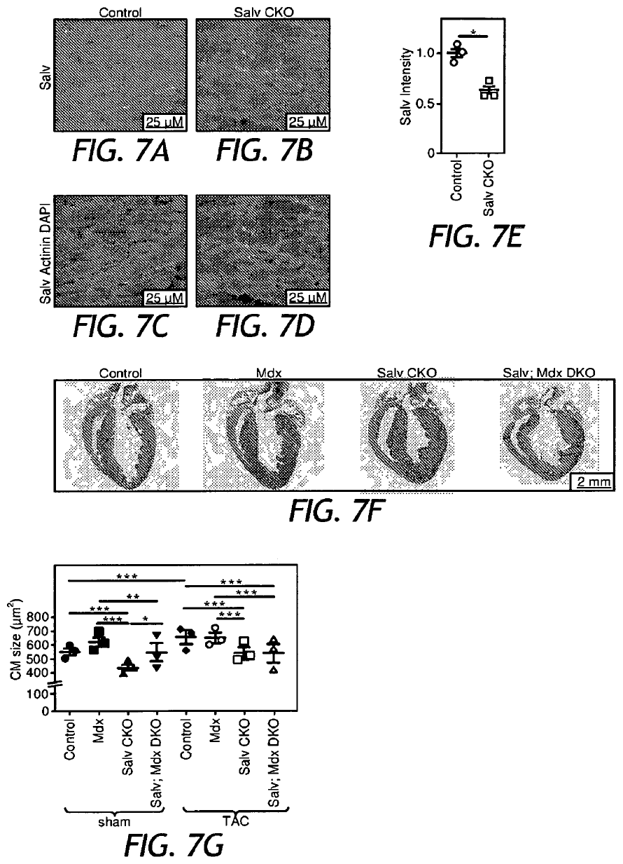

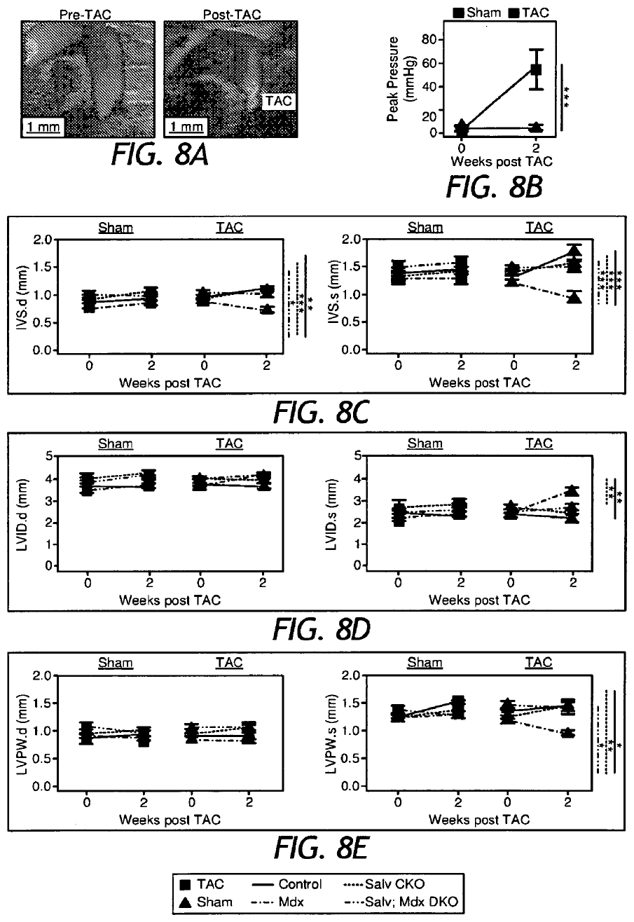

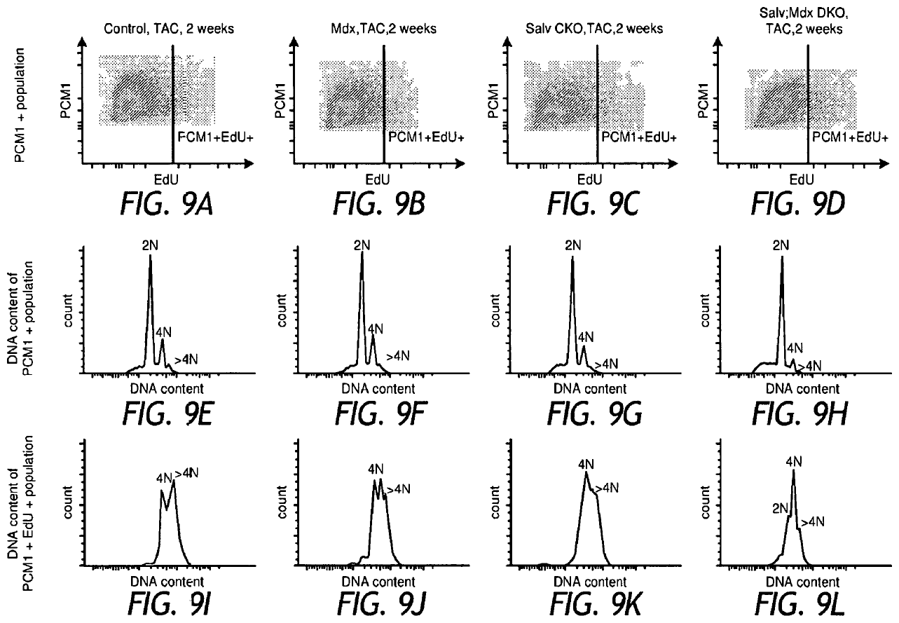

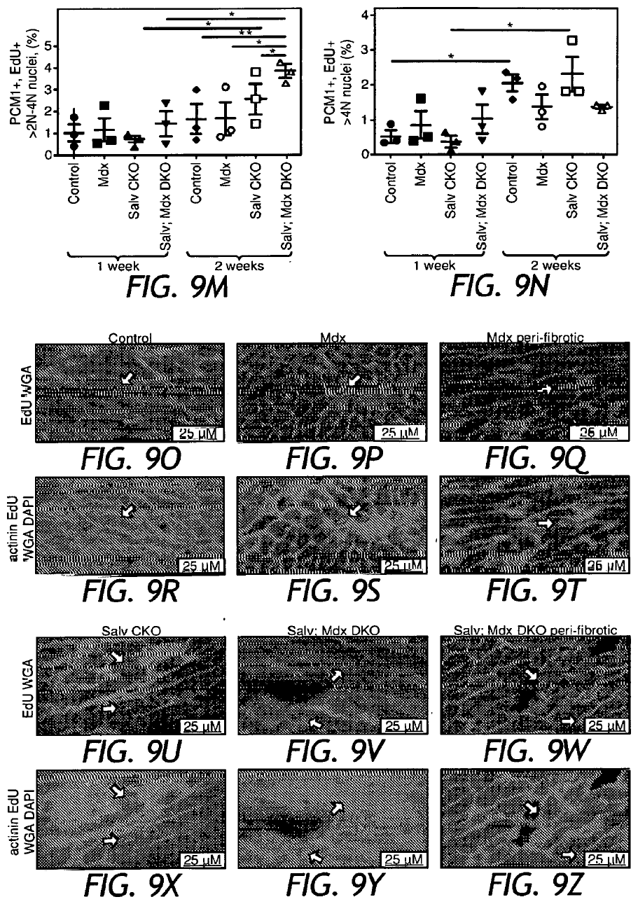

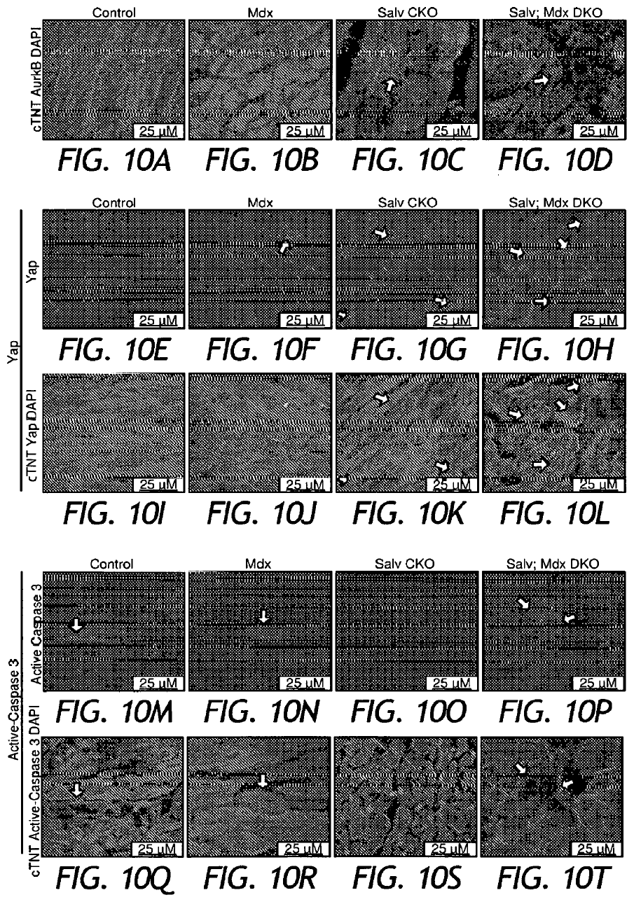

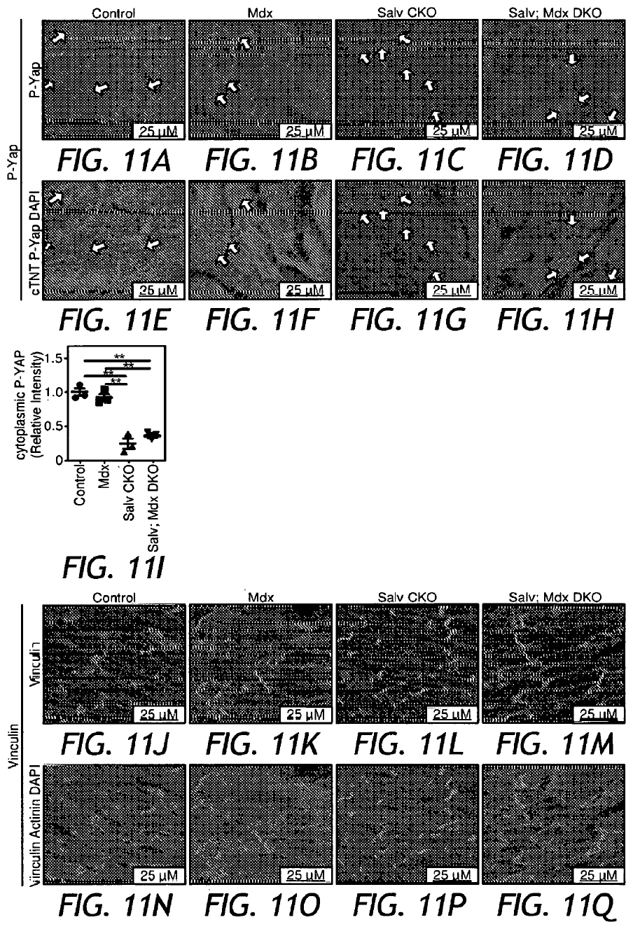

Dilated cardiomyopathy (DCM) with heart failure and myocardial fibrosis develops in DMD patients and approximately 10% of female carriers⁹. Mice were challenged with transverse aortic constriction (TAC)—an overload model of DCM with fibrosis, CM loss, and heart failure¹⁰ (FIGS. 7A-7G). Two weeks after TAC, Salv CKO and Salv;Mdx DKO CMs were smaller than control and Mdx CMs (FIG. 7G). Mdx hearts showed DCM with decreased heart function and increased fibrosis, indicating that Mdx hearts were prone to DCM (FIGS. 3A-3K; FIGS. 8A-8E)¹¹. In contrast, Salv;Mdx DKO hearts showed less severe dilation, less replacement fibrosis, and maintained cardiac function equivalent to that of control and Salv CKO hearts (FIGS. 3A-3K; FIGS. 8A-8E). Mdx mice challenged with TAC undergo CM loss¹¹. CM number was higher in Salv;Mdx DKO hearts than in Mdx hearts (FIG. 3L). Salv;Mdx DKO hearts showed increased numbers of EdU-positive diploid (2N) CM nuclei that were enriched in peri-fibrotic areas and aurkb expressing CM, indicating increased proliferation (FIGS. 3M, 3N, 3O; FIGS. 9A-9Z, 10A-10D). More CMs had nuclear Yap in Salv CKO and Salv;Mdx DKO hearts than in Mdx hearts (FIG. 3p; FIGS. 10E-10L). The higher number of CMs in Salv;Mdx DKO hearts than in Mdx hearts was partly because of reduced apoptosis, particularly one week after TAC (FIG. 3Q, FIGS. 10M-10T). Furthermore, cytoplasmic phosphorylated (P)-Yap levels were lower in Salv CKO and Salv;Mdx DKO CMs than in control and Mdx CMs (FIGS. 11A-11I). Unlike in the resection model, vinculin expression was comparable among the 4 genotypes (FIGS. 11J-11Q). These results suggest that suppression of Mdx cardiomyopathy by deleting Salv is due to increased CM proliferation and, immediately after TAC, to reduced apoptosis.

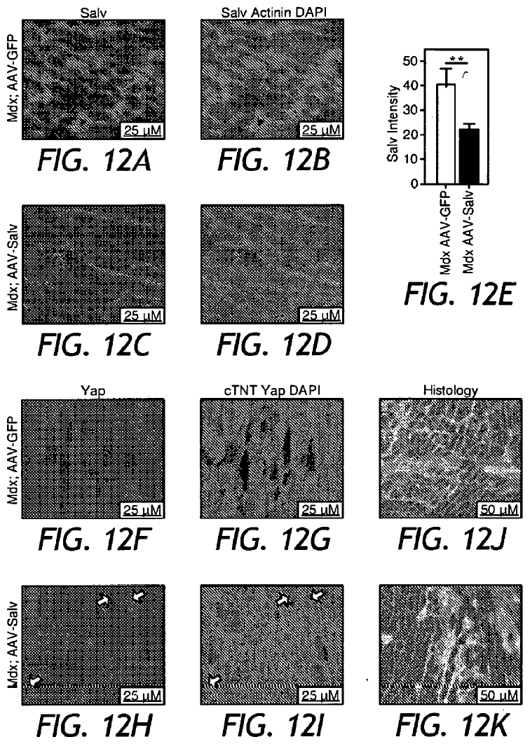

To investigate Hippo deficiency in Mdx C57BL/10J mice using gene therapy, the inventors performed TAC and injected an adeno-associated virus 9 (AAV9) encoding Salv shRNA or a control virus (FIG. 3R-3V; FIGS. 12A-12E)¹². Compared to the control knockdown, Salv knockdown preserved cardiac function, increased nuclear Yap and Yap target gene expression, and reduced fibrosis in TAC-challenged Mdx C57BL/10J hearts (FIG. 3R-3V; FIGS. 12F-12K). The mRNA levels of Yap target genes encoding proteins that regulate cell proliferation increased to a greater extent than did those of genes encoding cytoskeletal components (FIG. 3U), in specific embodiments reflecting differences in sensitivity to Yap.

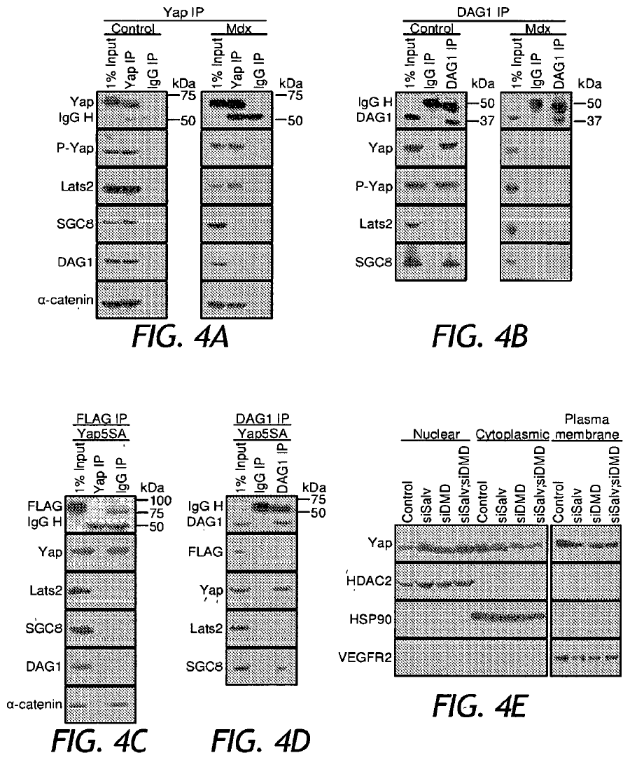

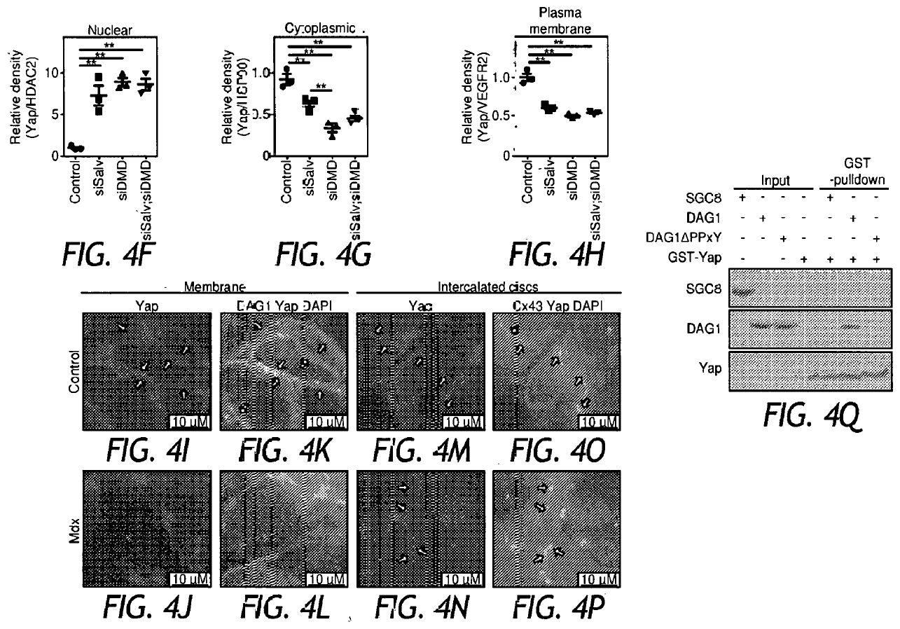

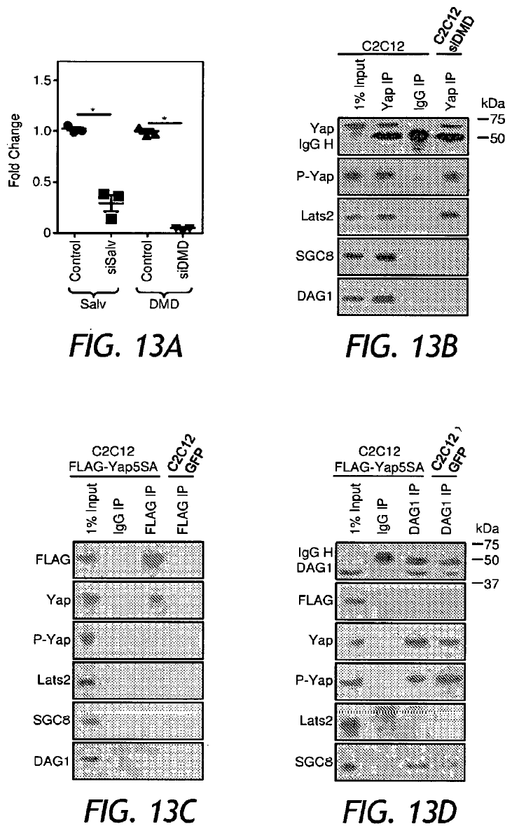

It was determined whether DGC complex components directly interact with Yap in control and Mdx P12 heart extracts. DAG1 contains a PPxY motif that binds WW domain-containing proteins, such as Yap and dystrophin¹³. Yap pulldowns revealed that Yap associated with DGC components Sgcδ and DAG1 in control but not Mdx extracts, whereas the interaction between Yap and Lats2, as well as an interaction between Yap and α-catenin, was detected in both control and Mdx extracts (FIG. 4A). DAG1 pulldowns revealed that DAG1 associated with Sgcδ and Yap in control but not Mdx extracts (FIG. 4B). Lats2 did not associate with DAG1, suggesting that Hippo pathway components did not form a complex with the DGC. Using the C2C12 myoblast cell line, Yap interacted with DGC components but that the interaction was disrupted when the gene encoding dystrophin (Dmd) was knocked down (FIG. 13A, 13B).

P-Yap was present in the DGC (FIG. 4B; FIG. 13D), indicating that Yap phosphorylation by Lats kinase promotes the interaction between Yap and the DGC, in specific embodiments. To test this, the interaction was examined between Yap and the DGC in cardiac extracts from transgenic mice expressing Flag-tagged Yap—Yap5SA—that is impervious to the Hippo kinase cascade in adult CMs¹⁴. Yap5SA did not interact with DGC components or Lats2 but did interact with α-catenin (FIGS. 4C, 4D). In Yap5SA-transfected C2C12 cells, neither DGC components nor Lats2 interacted with Yap5SA (FIGS. 13C, 13D). DAG1 interacted with endogenous Yap but not Yap5SA (FIG. 4D; FIG. 13D). Similar to what was observed in the heart, Lats2 and DAG1 did not interact in C2C12 cells (FIG. 13D).

C2C12 cell fractionation studies revealed that, in control cells, Yap was localized in the plasma membrane, cytoplasm, and nuclear fractions (FIGS. 4E-4H). Compared to control cells, Salv knockdown C2C12 cells and Salv;Dmd knockdown C2C12 cells had decreased Yap localization in the plasma membrane and cytoplasmic fractions and increased Yap localization in the nuclear fraction (FIGS. 4E-4H). In contrast to CMs, Dmd knockdown C2C12 cells had more nuclear Yap. A difference between C2C12 cells and CMs is the presence of the intercalated disc (ICD) in mature CMs. Because Yap binds α-catenin that localizes to the ICD in CMs, Yap binding to the ICD may compensate for DGC deficiency in CMs (FIG. 4A, 4C)¹⁵. Super resolution microscopy revealed that Yap colocalized with DAG1 in the plasma membrane in control but not Mdx CMs (FIG. 4J-4L). However, Yap colocalized in the ICD with connexin 43 (Cx43) in both control and Mdx CMs (FIGS. 4M-4P). In addition, experiments with purified GST fusion proteins revealed that Yap directly bound DAG1 through the DAG1 PPxY motif but did not directly interact with Sgcδ (FIG. 4Q). Therefore, Yap phosphorylation is not required for DAG1 binding in vitro. Collectively, these data suggest that Hippo phosphorylation of Yap promotes the Yap-DGC complex formation in vivo.

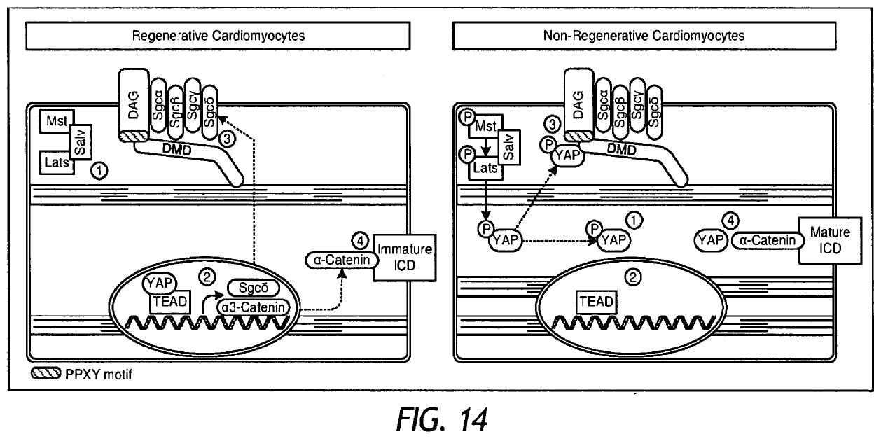

The DGC is a node for mechanical signaling. Mechanical signaling regulates Yap subcellular localization, but the mechanism is poorly understood¹⁶. The data reveal that the DGC sequesters P-Yap, perhaps to prevent the action of activating phosphatases, as a mechanism to regulate CM proliferation in the postnatal and adult heart (FIG. 14). The findings indicate exquisite coordination of extracellular and mechanical signaling with nuclear events in the context of a mechanically stressed CM. The Hippo-Yap-DGC negative regulatory loop is dysfunctional in the stressed CM, preventing an adequate proliferative response. The findings also indicate that Hippo signaling is maladaptive in DMD cardiomyopathy and raise the possibility that Hippo deficiency, in combination with other approaches such as gene editing, can be used to treat muscular dystrophy¹⁷⁻¹⁹

### Example 2

### Examples of Materials and Methods

Mice. Control (Mhy6-Cre^{ert}; mTmG), Mdx (Mdx;Mhy6-Cre^{er};mTmG), Salv CKO (Salv^{fx/fx};Mhy6-Cre^{ert};mTmG), and Salv;Mdx DKO (Salv^{fx/fx};Mdx;Mhy6-Cre^{ert};mTmG) mice were used for genetic studies. For studies involving AAV9 infection, C57-BL/OScSn-Dmd^{mdx}/J mice (The Jackson Laboratory, Bar Harbor, Me., USA) were used. Because the allele encoding dystrophin is X-linked, only male mice were used in this study. The gain-of-function transgenic Yap 5SA mouse line was created by injecting mice with the CAG-loxP-eGFP-Stop-loxP-Flag YAP2 5SA-IRES-βGal plasmid¹². The sequence of Flag-YAP2 5SA was obtained from the pCMV-Flag YAP2 5SA plasmid (a gift from Kun-Liang Guan (UCSD), Addgene plasmid #27371). Mhy6-Cre^{ert} mice were crossed with hemizygous Yap5SA mice. To induce the expression of Yap5SA in CMs, tamoxifen (150 mg) was injected daily for 4 days into 6-week-old Yap5SA;Mhy6-Cre^{ert} mice.

For each experimental group, a minimum of 3 mice were studied that were randomized by using block randomization. Sample sizes were estimated based on initial studies. Mice were used for these studies because they are amenable to genetic manipulation and can be made into models of human diseases. All animal protocols and procedures were approved by the Institutional Animal Care and Use Committee (IACUC) of Baylor College of Medicine in Houston, Tex. For all surgeries and echocardiographic studies, researchers were blinded from mouse genotypes.

Apex resection. Resection of the heart apex or sham surgery was performed at P8 in mice, as previously described⁴. Tamoxifen (50 mg) was injected daily from P7 to P10. Hearts were collected at 4 and 21 days after resection. For data in FIG. 1, Sham or apex resection was performed in nonregenerative-stage hearts (postnatal day 8) of control, Mdx, Salv conditional knockout (CKO), and Salv;Mdx double knockout (DKO) mice. All mice carried the Myh6-Cre^{ERT};mTmG allele, and tamoxifen was administered daily from P7 to P10.

Transverse aortic constriction (TAC). Mice were challenged with TAC because Mdx mice have a mild phenotype and develop cardiomyopathy after aging to approximately 15 months²⁰. On the day of surgery and 1 day after surgery, 9- to 10-week-old mice were injected with tamoxifen (150 mg). The thoracic aorta between the carotid arteries was constricted by using 6-0 silk thread between the carotid arteries. Mice that did not survive overnight from the surgery were excluded. Echocardiographic analysis was performed on the day before surgery and 2 weeks after the surgery. For the studies involving AAV9, echocardiography was performed prior to surgery and 2, 8, and 11 weeks after surgery. TAC and sham surgeries were performed in C57BL/10J Mdx mice. For knockdown studies, AAV-GFP (green fluorescent protein) or AAV-Salv virus was administered at the time of surgery. In FIGS. 3R-3S, Mdx sham groups prior to viral injection at week 0 were collapsed into one group (Sham pre-treatment).

Cardiac function analysis. Echocardiography was performed in the Baylor College of Medicine Mouse Phenotyping Core. Transthoracic echocardiography was performed by using the VisualSonics Vevo 2100 system equipped with a 40 Mhz 550s probe. Mice were anesthetized with 2% isoflurane inhalation (driven by 2 L/min oxygen) and placed on a heated platform (37° C.) with integrated physiologic monitoring capabilities. Two-dimensional B-mode imaging was used to capture the long-axis projection with guided M-Mode images, and a PW Doppler pulse wave was recorded for associated echocardiographic measurements. The average reading for each echocardiographic parameter was recorded from at least 3 distinct frames from each mouse.

AAV9 targeting Salv. The parental vector pENN.AAV.cTNT, p1967-Q was obtained from the University of Pennsylvania Vector Core. A triple miR30-flanked shRNA directed at Salv was cloned downstream of a green fluorescent protein (GFP) reporter. The subsequent construct was packed into AAV9 by the IDDRC Neuroconnectivity Core at Baylor College of Medicine. Mice were administered a single retro-orbital injection of virus with a total of 1×10¹² viral genomes delivered, and TAC or sham surgery was performed. Hearts were collected 13 weeks after the surgery.

Histologic analysis. For apex resection studies, hearts were collected 3 weeks after the surgery. For TAC genetic studies, hearts were collected 2 weeks after surgery. Hearts were fixed in 10% formalin, embedded into paraffin, and sectioned for further analysis. For AAV9 studies, hearts were collected 11 weeks after surgery; hearts were then frozen and sectioned for further analysis.

Trichrome staining was performed to determine the degree of heart injury or fibrosis. Fibrotic scarring stained blue, and scar size was measured by using ImageJ software. The size of the extra apex was determined by drawing a line at the site of resection and measuring the area outside of the line by using ImageJ software.

Immunohistochemistry. Paraffin-embedded and frozen tissues were used for immunohistochemical analysis. For paraffin-embedded sections, samples were deparaffinized and rehydrated, treated with 3% H₂O₂ in EtOH, treated with antigen retrieval solution (Vector Laboratories, Inc., Burlingame, Calif., USA), blocked with 10% donkey serum in phosphate-buffered saline (PBS), and then incubated with primary antibodies. For frozen sections, samples were fixed with acetone, treated with 0.3% H₂O₂ in PBS, treated with 0.1 M ammonium chloride, blocked with 10% donkey serum in PBS, and then incubated with primary antibodies. Primary antibodies were detected with fluorescently labeled secondary antibodies. Primary antibodies used in this study were as follows: mouse anti-cardiac troponin T (Thermo Fisher Scientific Inc., Waltham, Mass., USA), mouse anti-vinculin (Thermo Fisher), mouse anti-ß-dystroglycan (Santa Cruz Biotechnology, Inc., Dallas, Tex., USA), mouse anti-Salvador (Santa Cruz), rabbit anti-Yap1 (Novus Biologicals, LLC, Littleton, Colo., USA), rabbit anti-phospho Yap (Cell Signaling Technology, Inc., Danvers, Mass., USA), rabbit anti-aurora kinase B (Abcam, Cambridge, UK), rabbit anti-sarcomeric actinin (Abcam), rabbit anti-CCNE2 (Abcam), mouse anti-Talin (Sigma-Aldrich Co. LLC., St. Louis, Mo., USA), mouse anti-connexin 43 (Santa Cruz), rabbit anti-active caspase 3 (Abcam), and rabbit anti-phosphohistone H3 (PHH3) (Abcam). Secondary antibodies used in this study were as follows: Alexa-546- or Alexa-647-conjugated anti-rabbit (Thermo Fisher), biotin-conjugated anti-mouse (Vector), and Alexa-488-conjugated streptavidin (Thermo Fisher). Immunostained samples were analyzed by using a Leica TCS SP5 confocal microscope. Images were processed by using Leica LAS AF software. Images from 3 different sections were documented for each sample.

For the quantification of CMs positive for AurkB, Yap, active caspase 3, and CCNE2, positive-staining CMs were quantified manually by using ImageJ software. The observer was blinded to genotypes. The quantification of CMs positive for Talin, Salv, and P-Yap was performed by measuring pixel intensity in the CM cytoplasm. When images were captured, intensity was normalized to the level of staining in fibroblasts.

Deconvolution epifluorescence microscopy was performed in the Baylor College of Medicine Integrated Microscopy Core by using an OMX-BLAZE 3D structured illumination microscope (GE Healthcare Life Systems, Pittsburgh, Pa., USA). Images were acquired with an Olympus PlanApo 60×/1.42 objective, in z-stacks with 0.125 □m spacing covering most of the tissue thickness. Images were deconvolved by using a conservative algorithm. The maximum intensity projected and histogram were adjusted by using SoftWorX 6.5.2. Three-dimensional volumes were also generated with the same software.

To quantify the number and size of CMs, cells were delineated by using wheat germ agglutinin (WGA) conjugated with Alexa 647 (Thermo Fisher). Tissues were costained with anti-cardiac troponin T to label CMs, and images were captured under a fluorescent microscope. The number and size of CMs were quantified manually by using ImageJ software. The observer was blinded to genotypes.

In CMs stained for cTNT, sarcomere length was measured manually by using ImageJ software. The observer was blinded to genotypes. CM orientation angles were measured manually by using ImageJ and were referenced to the plane of resection.

EdU incorporation analysis. For EdU incorporation studies on P8 resection models, 0.25 mg of EdU (5-ethynly-2-deoxyurindine) was injected into apex-resected animals at 4 hr before harvest on 4 days after resection. EdU incorporation was analyzed in paraffin-embedded tissues by using a Click-it EdU Imaging Kit (Thermo Fisher), followed by staining for cTNT and WGA. The number of EdU-positive nuclei was counted manually by using ImageJ software. The observer was blinded to genotypes.

For EdU incorporation studies on adult TAC models, 100 μg/g mouse of EdU was injected daily, starting at 2 days after surgery until 1 day before harvest. Hearts were harvested and nuclei were isolated for flow analysis according to previously described^{21,22}. In brief, nuclei were stained with PCM1 antibody (Sigma-Aldrich) and for EdU using Click-it EdU Flow Cytometry Assay Kit (Thermo Fisher). Analysis of nuclei was performed at Texas Heart Institute Flow Cytometry Core Facility using BD FACSAria (BD Biosciences). For EdU performed on sections, peri-fibrotic area was defined as within 350 μm from the replacement fibrosis that was only observed in Mdx and Salv;Mdx DKO hearts.

Collagen gel assay. A collagen gel assay was used to analyze P10 hearts, as previously described⁶. Gels were stained for cTNT and with DAPI to detect the migration of CMs to the bottom gel.

Quantitative real-time (RT)-PCR. Total RNA was extracted from frozen heart tissue by using the RNAeasy Micro kit (Qiagen, Hilden, Germany). Quantitative RT-PCR was performed as previously described⁶ by using the StepOnePlus Real-Time PCR System (LifeTechnologies).

Immunoprecipitation and Western blotting. Immunoprecipitation and Western blotting were performed as previously described,⁵ with the following minor modifications. Hearts were treated with modified radioimmunoprecipitation assay (RIPA) lysis buffer (50 mM Tris-HCl, pH 7.4, 50 mM NaCl, 1 mM EDTA, 0.2% sodium deoxycholate, 0.05% sodium dodecyl sulfate [SDS], 0.2% Triton X-100, 0.5% NP-40) supplemented with complete protease and phosphatase inhibitors (The Roche Group, Basel, Switzerland).

Protein concentration was determined by using a Qubit Protein Assay Kit on a Qubit 3.0 Fluorometer (Thermo Fisher). Primary antibodies used for immunoprecipitation were as follows: anti-Yap1 (5 g; Novus) and anti-DAG1 (3 g; Abcam). Anti-Flag M2 affinity gel (Sigma-Aldrich) was used for Flag pulldown assays. Primary antibodies used for immunoblotting were as follows: anti-Yap1 (1:1000 dilution; Novus), anti-phospho-Yap (Ser127) (1:1000; Cell Signaling), anti-LATS2 (1:500 dilution; Bethyl Laboratories), anti-SGCD (1:500 dilution; Santa Cruz), anti-DAG1 (1:500 dilution; Abcam), anti-α-catenin (1:50000 dilution; Abcam), and anti-Flag (1:1000 dilution; Sigma-Aldrich).

For the Yap5SA studies, expression of Yap5SA in CMs was induced by tamoxifen injection starting 5 days before harvest for 4 consecutive days. For studies performed in C2C12 cells, subcellular protein lysate fractions were collected from differentiated C2C12 cells after siRNA-mediated knockdown of Salv and/or the gene encoding dystrophin (Dmd).

Cell Culture. C2C12 cells (ATCC CRL-1772) were cultured in Dulbecco's modified Eagle medium (D-MEM; Thermo Fisher) supplemented with 20% fetal bovine serum (HyClone, GE Healthcare Life Systems) and 1× penicillin/streptomycin (Thermo Fisher). Cells were tested for mycoplasma by using a Myco Probe mycoplasma detection kit (R&D Systems). To induce differentiation, confluent cells were treated with differentiation media containing D-MEM supplemented with 2% horse serum (Thermo Fisher) and 1× penicillin/streptomycin.

For immunoprecipitation studies, cells were differentiated for 7 days. For Dmd knockdown studies, Lipofectamine RNAiMax (Thermo Fisher) was used to transfect siRNA into differentiated cells 2 days before harvest. Yap5SA (pCMV-Flag Yap2) or GFP plasmid was transfected into C2C12 cells by using Lipofectamine 3000 (Thermo Fisher) one day before the differentiation treatment was started. For subcellular fractionation, cells were differentiated for 3 days and treated with siRNA at day 1 of differentiation for 48 hours before harvest. Nuclear, cytoplasmic, and plasma membrane fractions of cellular extracts were prepared by using a subcellular protein fractionation kit for tissues (Thermo Fisher) according to the manufacturer's instructions.

Recombinant GST-fusion proteins and binding assays. The GST-YAP1 plasmid was a kind gift from Stefano Piccolo (University of Padova, Italy). GST-DAG1 (ß-dystroglycan domain, amino acids 652-893 of DAG1) and GST-SGCD (full-length, amino acids 1-289) were created by Gibson Assembly DNA cloning (NEB, Ipswich, Mass., USA) by using synthesized gBlocks gene fragments (IDT) for in-frame fusion with the GST moiety of pGEX-4T-2. To generate the DAG1ΔPPxY mutant, the C-terminal PPxY domain (amino acids 887-890) of DAG1 was deleted by using PCR-based site-directed mutagenesis with the NEBaseChanger tool and the Q5 Site-Directed Mutagenesis kit (NEB). All constructs were confirmed by DNA sequencing. Recombinant GST proteins were expressed in the E. coli strain BL21 (DE3) (ThermoFisher) by induction with 1 mM isopropyl 3-D-1-thiogalactopyranoside (IPTG) for 24 hours. Bacterial pellets were lysed in GST lysis buffer (1×PBS, 20 mM HEPES, 1 mM EGTA, 0.2% Triton X-100) supplemented with cOmplete, Mini, EDTA-free Protease Inhibitor and PhosSTOP phosphatase inhibitor cocktails (Sigma-Aldrich). After brief sonication of the lysates, 50% glutathione-S-transferase resin (GE Healthcare) was added, and samples were rocked at 4° C. for 4 hours for the affinity purification of GST-tagged proteins.

For in vitro binding assays, purified GST proteins (500 mM) were treated with thrombin protease (GE Healthcare) to remove the GST tag. Cleaved proteins were then mixed with GST-YAP1 or GST-DMD bound to glutathione beads and were rocked over ice for 3 hours. Beads were washed three times by rocking in PBS+20 mM HEPES with 0.2% Triton X-100 at 4° C. for 5 minutes and were pelleted. Samples were separated by using SDS-polyacrylamide gel electrophoresis, transferred to PVDF membranes, and analyzed via Western blotting.

Estimation of Cardiomyocyte Numbers on Sections. For each section, total number of CMs was estimated from the number of CMs in 0.1 mm² area multiplied by the total area.

Statistical Analysis. The n number for each studies and analysis used in each panel are stated in each section of figure legends. All bar graphs except for Extended Data FIG. 1o represent mean+/−s.e.m.

### REFERENCES

1 Xin, M., Olson, E. N. & Bassel-Duby, R. Mending broken hearts: cardiac development as a basis for adult heart regeneration and repair. Nature reviews. Molecular cell biology 14, 529-541, doi:10.1038/nrm3619 (2013).
2 Porrello, E. R. et al. Transient regenerative potential of the neonatal mouse heart. Science (New York, N.Y.) 331, 1078-1080, doi:10.1126/science.1200708 (2011).
3 Bergmann, O. et al. Dynamics of Cell Generation and Turnover in the Human Heart. Cell 161, 1566-1575, doi:10.1016/j.cell.2015.05.026 (2015).
4 Heallen, T. et al. Hippo signaling impedes adult heart regeneration. Development 140, 4683-4690 (2013).
5 Heallen, T. et al. Hippo pathway inhibits Wnt signaling to restrain cardiomyocyte proliferation and heart size. Science (New York, N.Y.) 332, 458-461, doi:10.1126/science.1199010 (2011).
6 Morikawa, Y. et al. Actin cytoskeletal remodeling with protrusion formation is essential for heart regeneration in Hippo-deficient mice. Sci. Signal. 8, ra41 (2015).
7 Ryder-Cook, A. S. et al. Localization of the mdx mutation within the mouse dystrophin gene. The EMBO journal 7, 3017-3021 (1988).
8 Houtchens, G. R., Foster, M. D., Desai, T. A., Morgan, E. F. & Wong, J. Y. Combined effects of microtopography and cyclic strain on vascular smooth muscle cell orientation. Journal of biomechanics 41, 762-769, doi:10.1016/j.jbiomech.2007.11.027 (2008).
9 van Westering, T. L., Betts, C. A. & Wood, M. J. Current understanding of molecular pathology and treatment of cardiomyopathy in duchenne muscular dystrophy. Molecules (Basel, Switzerland) 20, 8823-8855, doi:10.3390/molecules20058823 (2015).
10 Kassiri, Z. et al. Combination of tumor necrosis factor-alpha ablation and matrix metalloproteinase inhibition prevents heart failure after pressure overload in tissue inhibitor of metalloproteinase-3 knock-out mice. Circulation research 97, 380-390, doi:10.1161/01.RES.0000178789.16929.cf (2005).
11 Kamogawa, Y. et al. Dystrophin-deficient myocardium is vulnerable to pressure overload in vivo. Cardiovasc. Res. 50, 509-515 (2001).
12 Leach, J. & Martin, J. F. Hippo Pathway Deficiency Reverses Systolic Heart Failure in Preparation (2016).
13 Tommasi di Vignano, A., Di Zenzo, G., Sudol, M., Cesareni, G. & Dente, L. Contribution of the different modules in the utrophin carboxy-terminal region to the formation and regulation of the DAP complex. FEBS letters 471, 229-234 (2000).
14 Zhao, B., Li, L., Tumaneng, K., Wang, C. Y. & Guan, K. L. A coordinated phosphorylation by Lats and CK1 regulates YAP stability through SCF(beta-TRCP). Genes & development 24, 72-85, doi:10.1101/gad.1843810 (2010).
15 Li, J. et al. Alpha-catenins control cardiomyocyte proliferation by regulating Yap activity. Circulation research 116, 70-79, doi:10.1161/circresaha.116.304472 (2015).
16 Halder, G., Dupont, S. & Piccolo, S. Transduction of mechanical and cytoskeletal cues by YAP and TAZ. Nature reviews. Molecular cell biology 13, 591-600, doi:10.1038/nrm3416 (2012).
17 Long, C. et al. Postnatal genome editing partially restores dystrophin expression in a mouse model of muscular dystrophy. Science (New York, N.Y.) 351, 400-403, doi:10.1126/science.aad5725 (2016).
18 Nelson, C. E. et al. In vivo genome editing improves muscle function in a mouse model of Duchenne muscular dystrophy. Science (New York, N.Y.) 351, 403-407, doi:10.1126/science.aad5143 (2016).
19 Tabebordbar, M. et al. In vivo gene editing in dystrophic mouse muscle and muscle stem cells. Science (New York, N.Y.) 351, 407-411, doi:10.1126/science.aad5177 (2016).
20 McGreevy, J. W., Hakim, C. H., McIntosh, M. A. & Duan, D. Animal models of Duchenne muscular dystrophy: from basic mechanisms to gene therapy. Dis Model Mech 8, 195-213, doi:10.1242/dmm.018424 (2015).
21 Alkass, K. et al. No Evidence for Cardiomyocyte Number Expansion in Preadolescent Mice. Cell 163, 1026-1036, doi:10.1016/j.cell.2015.10.035 (2015).
22 Richardson, G. D. Simultaneous Assessment of Cardiomyocyte DNA Synthesis and Ploidy: A Method to Assist Quantification of Cardiomyocyte Regeneration and Turnover. Journal of visualized experiments: JoVE, doi:10.3791/53979 (2016).

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A method of regenerating cardiomyocytes, comprising the step of exposing cardiomyocytes to an effective amount of one or more agents that inhibit one or more of the following:
(a) DAG1;
(b) Yap;
(c) the binding of DAG1 to Yap; or
(d) the phosphorylation of Yap.

2. The method of claim 1, wherein the agent is a peptide, protein, nucleic acid, small molecule, or combination thereof.

3. The method of claim 1, wherein the exposing step occurs ex vivo and/or in vivo in a first individual.

4. The method of claim 3, wherein when the exposing step occurs ex vivo, the cardiomyocytes are from a first individual and the cardiomyocytes to which the agent(s) are exposed come from the first individual.

5. The method of claim 3, wherein when the providing step occurs ex vivo, the cardiomyocytes are from the first individual and the cardiomyocytes to which the agent(s) is provided come from a second individual different than the first individual.

6. The method of claim 3, wherein the first individual has a muscular or cardiac condition.

7. The method of claim 6, wherein the muscular condition is muscular dystrophy, fibrosis, myotonic dystrophy, myocarditis, heart failure, dilated cardiomyopathy, or a combination thereof.

8. The method of claim 6, wherein the cardiac condition is cardiovascular disease, cardiomyopathy, heart failure, myocardial infarction, ischemia, fibrosis, and/or necrosis.

9. The method of claim 8, wherein the cardiomyopathy is diabetic cardiomyopathy or age-related cardiomyopathy.

10. The method of claim 3, wherein the individual is provided an effective amount of one or more therapies for a muscular condition and/or a cardiac condition.

11. A method of treating an individual for a muscular or cardiac condition, comprising the step of:
(a) providing to the individual an effective amount of cardiomyocytes that have been exposed to one or more agents that inhibit one or more of the following:
(1) DAG1;
(2) Yap;
(3) the binding of DAG1 to Yap; or
(4) the phosphorylation of Yap; and/or
(b) exposing cardiomyocytes in vivo to one or more of the agents that inhibit one or more of (1), (2), (3), or (4).

12. The method of claim 11, wherein the agent is a peptide, protein, nucleic acid, small molecule, or combination thereof.

13. The method of claim 11, wherein the cardiomyocytes to which the one or more agents are provided are from the individual.

14. The method of claim 11, wherein the cardiomyocytes to which the one or more agents are provided are from a different individual than the individual to which the cardiomyocytes are provided.

15. The method of claim 11, wherein the individual has a muscular or cardiac condition.

16. The method of claim 15, wherein the muscular condition is muscular dystrophy, fibrosis, myotonic dystrophy, myocarditis, heart failure, dilated cardiomyopathy, or a combination thereof.

17. The method of claim 15, wherein the cardiac condition is cardiovascular disease, cardiomyopathy, heart failure, myocardial infarction, ischemia, fibrosis, and/or necrosis.

18. The method of claim 17, wherein the cardiomyopathy is diabetic cardiomyopathy or age-related cardiomyopathy.

19. The method of claim 11, wherein the individual is provided an effective amount of one or more therapies for a muscular or cardiac condition.
